(19) United States
(12) Patent Application Publication
(10) Pub. No.: US 2023/0358746 A1
(43) Pub. Date: Nov. 9, 2023

(54) METHODS AND KITS FOR DETERMINING SEROSTATUS OF SARS-CoV-2

(71) Applicant: Rational Vaccines, Inc., Cambridge, MA (US)

(72) Inventors: Agustin FERNANDEZ, Cambridge, MA (US); Edward GERSHBURG, Cambridge, MA (US); Paul PREDKI, Cambridge, MA (US)

(73) Assignee: Rational Vaccines, Inc., Cambridge, MA (US)

(21) Appl. No.: 18/030,154

(22) PCT Filed: Oct. 6, 2021
(86) PCT No.: PCT/US2021/053794

§ 371 Date: Apr. 4, 2023

Related U.S. Application Data
(60) Provisional application No. 63/164,894, filed on Mar. 23, 2021.
(60) Provisional application No. 63/088,035, filed on Oct. 6, 2020.

(51) Int. Cl. G01N 33/569; G01N 33/58; G01N 21/64

(52) U.S. Cl. CPC G01N 33/56983; G01N 33/582; G01N 21/6428; G01N 2333/165; G01N 2469/20; G01N 2021/6439

(57) ABSTRACT

The disclosure provides an assay for detecting if a subject is seropositive for SARS-CoV-2, or not, comprising incubating a biological sample from the subject with uninfected cells and SARS-CoV-2-infected cells, where the uninfected cells and the SARS-CoV-2-infected cells are distinguishable from each other, to form an admixture, and determining an amount of antibodies in the biological sample bound to the uninfected cells and to the SARS-CoV-2-infected cells, thereby determining whether the subject whose biological sample was tested is seropositive for SARS-CoV-2, or not.

### RELATED APPLICATIONS

This application claims priority to U.S. Provisional Patent Appl. No. 63/164,894, filed Mar. 23, 2021, and U.S. Provisional Patent Appl. No. 63/088,035, filed Oct. 6, 2020, each of which is incorporated herein by reference in its entirety.

### TECHNICAL FIELD

The disclosure relates to assays and methods for testing for viral infection, including biological (e.g., serological) sample assays to determine if a subject is seropositive or seronegative for SARS-CoV-2.

### BACKGROUND

COVID-19 is a new disease caused by a strain of coronavirus called severe acute respiratory syndrome coronavirus 2 (SARS-CoV-2). As of 10 Jul. 2020, over 12 million cases of COVID-19 have been reported, including over 500,000 deaths. Given the high rate of infectivity and mortality associated with SARS-CoV-2, there is clearly a need for rapid, sensitive, and reliable tests to determine whether a subject has been infected with SARS-CoV-2 infection, and/or whether a subject is seropositive for SARS-CoV-2. The present disclosure meets this need by providing a biological sample (e.g., serological) assay for SARS-CoV-2 that quickly and accurately determines whether or not a subject is seropositive for SARS-CoV-2.

### SUMMARY

The present disclosure provides methods and kits for determining whether a subject comprises antibodies to SARS-CoV-2.

In a first aspect, the disclosure provides a method for determining whether a subject is seropositive for SARS-CoV-2 or not, comprising (optionally in the following order): a) admixing a biological sample obtained from the subject with test cells, wherein the biological sample comprises antibodies from the subject, and wherein the test cells comprise: (i) fixed and permeabilized test cells uninfected with SARS-CoV-2 virus; and (ii) fixed and permeabilized test cells infected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a first admixture comprising the biological sample and the fixed and permeabilized test cells, and maintaining said first admixture under conditions and for a time period sufficient to permit the antibodies to bind to antigens present in the fixed and permeabilized test cells; b) removing the biological sample from said first admixture, wherein the biological sample comprises remaining antibodies from the subject unbound to the fixed and permeabilized test cells; c) admixing a detection antibody with the fixed and permeabilized test cells, wherein the detection antibody comprises a detectable label, thereby generating a second admixture comprising the detection antibody and the fixed and permeabilized test cells, and maintaining said second admixture for a time period sufficient to permit the detection antibody to bind to the antibodies bound to the fixed and permeabilized test cells; d) washing said second admixture to remove unbound detection antibody from the fixed and permeabilized test cells; and e) measuring: (i) an amount of detection antibody bound to the fixed and permeabilized cells uninfected with SARS-CoV-2 virus; and (ii) an amount of detection antibody bound to the fixed and permeabilized cells infected with SARS-CoV-2 virus, thereby determining whether the subject is seropositive for SARS-CoV-2 or not, wherein if the amount of detection antibody measured in (e)(ii) is greater than the amount of detection antibody measured in (e)(i), and the amount of detection antibody measured in (e)(ii) meets or exceeds a pre-determined positive value or a calculated positive value, the subject is determined seropositive for SARS-CoV-2. In particular embodiments of any of the methods disclosed herein, the pre-determined or calculated positive value is a normalized value, e.g., the ratio of the mean signal on positive cells to the mean signal on negative cells, and the signal determined for the subject is also a normalized value, e.g., the ratio of the subject's signal on positive control cells to the subject's signal on negative control cells. In some embodiments, a biological sample comprises an antibody-containing biological sample. In some embodiments, an antibody-containing biological sample comprises serum, blood, salivary secretions (e.g., saliva), lacrimal secretions, respiratory secretions, or intestinal secretions.

In a related second aspect, the disclosure provides a method for determining whether a subject is seropositive for SARS-CoV-2 or not, comprising (optionally in the following order): a) admixing a biological sample obtained from the subject with fixed and permeabilized control cells comprising pathogen antigens other than SARS-CoV-2-specific antigens, thereby generating a first admixture; and maintaining said first admixture for a time period sufficient to permit the cross-reactive antibodies in virus the biological sample to bind to the pathogen antigens present in the fixed and permeabilized control cells, thereby experiencing pre-adsorption; b) collecting the biological sample from the first admixture, wherein the biological sample has been depleted of SARS-CoV-2 cross-reactive antibodies; c) admixing the biological sample with test cells, wherein the biological sample comprises antibodies from the subject depleted of SARS-CoV-2 cross-reactive antibodies, and wherein the test cells comprise: (i) fixed and permeabilized test cells uninfected with SARS-CoV-2 virus; and (ii) fixed and permeabilized test cells infected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a second admixture comprising the biological sample and the fixed and permeabilized test cells, and maintaining said second admixture under conditions and for a time period sufficient to permit the antibodies to bind to antigens present in the fixed and permeabilized test cells; d) removing the biological sample from said second admixture, wherein the biological sample comprises remaining antibodies from the subject unbound to the fixed and permeabilized test cells; e) admixing a detection antibody with the fixed and permeabilized test cells, wherein the detection antibody comprises a detectable label, thereby generating a third admixture comprising the detection antibody and the fixed and permeabilized test cells, and maintaining said third admixture for a time period sufficient to permit the detection antibody to bind to the antibodies bound to the fixed and permeabilized test cells; f) washing said third admixture to remove unbound detection antibody from the fixed and permeabilized test cells; and g) measuring: (i) an amount of detection antibody bound to the fixed and permeabilized cells uninfected with SARS-CoV-2 virus; and (ii) an amount of detection antibody bound to the fixed and permeabilized cells infected with SARS-CoV-2 virus, thereby determining whether the subject is seropositive for SARS-CoV-2 or not, wherein if the amount of detection antibody measured in (g)(ii) is greater than the amount of detection antibody measured in (g)(i), and the amount of detection antibody measured in (g)(ii) meets or exceeds a pre-determined positive value or a calculated positive value, the subject is determined seropositive for SARS-CoV-2. In certain embodiments, the fixed and permeabilized control cells comprise cells infected with influenza A virus, influenza B virus, hepatitis C virus, hepatitis B virus, Haemophilus influenza virus, alpha coronavirus 229E, alpha coronavirus NL63, beta coronavirus OC43, beta coronavirus HKU1, or respiratory syncytial virus, or any combination thereof. In certain embodiments, the fixed and permeabilized control cells comprise antigens cross-reactive with SARS-CoV-2. In some embodiments, a biological sample comprises an antibody-containing biological sample. In some embodiments, an antibody-containing biological sample comprises serum, blood, salivary secretions, lacrimal secretions, respiratory secretions, or intestinal secretions.

In various embodiments of any of the aspects disclosed, the fixed and permeabilized test cells uninfected with SARS-CoV-2 and the fixed and permeabilized test cells infected with SARS-CoV-2 are distinguishable from each other by fluorescence emission when irradiated with wavelengths of light that excite an exogenously-introduced fluorescent colorant. In certain embodiments, the exogenously-introduced fluorescent colorant comprises a dye, optionally an intracellularly fluorescent molecule. In certain embodiments, the exogenously-introduced fluorescent colorant forms a covalent linkage with intracellular molecules in the fixed and permeabilized test cells. In certain embodiments, said exogenously-introduced fluorescent colorant comprises caroxyfluorescein succinimidyl ester (CFSE). In certain embodiments, either one, but not both, of: (i) the fixed and permeabilized test cells uninfected with SARS-CoV-2; or (ii) the fixed and permeabilized cells test cell infected with SARS-CoV-2 comprise the exogenously-introduced fluorescent colorant. In certain embodiments, (i) the fixed and permeabilized test cells uninfected with SARS-CoV-2; and (ii) the fixed and permeabilized test cells infected with SARS-CoV-2 each comprise a different exogenously-introduced fluorescent colorant by which the two fixed and permeabilized test cells are distinguishable from each other according to the intensity of fluorescence in a defined emission spectrum, and which is distinguishable from the fluorescence emission spectrum of any other fluorophores used in the assay.

In various embodiments of any of the aspects disclosed, the antigens present in the fixed and permeabilized test cells that bind the antibodies present in the biological sample (e.g., serum sample) are measured by incubating said antigens bound to antibodies with labeled anti-human antibodies to form labeled antigen-antibody complexes. In certain embodiments, said labeled anti-human antibodies are labeled with a compound whose fluorescence emission spectrum is distinguishable from the fluorescence emission spectrum of any other fluorophores utilized in the assay. In certain embodiments, the label of said labeled anti-human antibodies comprises an antibody-linked fluorescent molecule or fluorophore. In certain embodiments, said labeled anti-human antibodies comprises allophycocyanin (APC) conjugated anti-human IgG.

In a further related third aspect, the disclosure provides a biological sample (e.g., serum sample) assay kit for determining whether a subject is seropositive for SARS-CoV-2 or not according to the method of any one of claims 1-14, comprising: a) one or more vessels collectively comprising: (i) fixed and permeabilized test cells uninfected with SARS-CoV-2 virus; and (ii) fixed and permeabilized test cells infected with SARS-CoV-2 virus, wherein the fixed and permeabilized test cells uninfected with SARS-CoV-2 and the fixed and permeabilized test cells infected with SARS-CoV-2 are distinguishable from each other; and b) a second vessel comprising: (iii) fixed and permeabilized control cells comprising pathogen antigens other than SARS-CoV-2-specific antigens. In certain embodiments, the fixed and permeabilized control cells are infected with one or more of: influenza A virus, influenza B virus, hepatitis C virus, hepatitis B virus, Haemophilus influenza virus, alpha corona virus 229E, alpha coronavirus NL63, beta coronavirus OC43, beta coronavirus HKU1, or respiratory syncytial virus, or any combination thereof. In certain embodiments, the fixed and permeabilized control cells comprise antigens cross-reactive with SARS-CoV-2. In certain embodiments, the kit also includes a third vessel comprising labeled anti-human antibodies. In certain embodiments, the label of said labeled anti-human antibodies comprises a compound that when irradiated provides a fluorescence emission spectrum distinguishable from that of any other fluorophores utilized in the assay. In certain embodiments, the labeled anti-human antibodies comprise allophycocyanin (APC) conjugated anti-human IgG. In certain embodiments, the kit also includes a fourth vessel comprising a calibrating solution respective to the type of biological sample being analyzed. In some embodiments, the calibrating solution comprises a calibrating biological sample or biological sample-mimic. In certain embodiments, the calibrating solution comprises serum or serum-mimic. In certain embodiments, the calibrating solution comprises saliva or saliva-mimic. In certain embodiments, the kit includes a fifth vessel comprising a positive biological sample control. In certain embodiments, the positive biological sample control comprises SARS-CoV-2-specific antibodies. In certain embodiments, the kit includes a sixth vessel comprising a negative biological sample control. In certain embodiments, the negative biological sample control does not comprise SARS-CoV-2-specific antibodies. In another embodiment, the negative biological sample control comprises antibody-free biological sample. In certain embodiments, the fixed and permeabilized test cells uninfected with SARS-CoV-2 and the fixed and permeabilized test cells infected with SARS-CoV-2 are distinguishable from each other by fluorescence emission when irradiated with wavelengths of light that excite an exogenously-introduced fluorescent colorant. In certain embodiments, the exogenously-introduced fluorescent colorant comprises a dye, optionally an intracellularly fluorescent molecule. In certain embodiments, the exogenously-introduced fluorescent colorant forms a covalent linkage with intracellular molecules in the fixed and permeabilized test cells. In certain embodiments, said exogenously-introduced fluorescent colorant comprises caroxyfluorescein succinimidyl ester (CFSE). In certain embodiments, either one, but not both, of: (i) the fixed and permeabilized test cells uninfected with SARS-CoV-2; or (ii) the fixed and permeabilized test cells infected with SARS-CoV-2 comprise an exogenously-introduced fluorescent colorant. In certain embodiments, (i) the fixed and permeabilized test cells uninfected with SARS-CoV-2 and (ii) the fixed and permeabilized test cells infected with SARS-CoV-2 each comprise a different exogenously-introduced fluorescent colorant by which the two fixed and permeabilized test cells are distinguishable from each other according to the intensity of fluorescence in a defined emission spectrum, and which is distinguishable from the fluorescence emission spectrum of any other fluorophores used in the assay.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIG. 1 is a diagram illustrating an embodiment of the SARS-CoV-2 biological sample assay disclosed herein, which depicts the preparation of fixed and permeabilized test cells, and the assaying of patient serum.

### DETAILED DESCRIPTION

A new disease called coronavirus disease 2019 (COVID-19) recently emerged globally (Centers for Disease Control (CDC), 2020). COVID-19 illnesses have ranged from mild symptoms to severe illness and death. The World Health Organization declared the outbreak a Public Health Emergency of International Concern on 30 Jan. 2020, and a pandemic on 11 Mar. 2020. As of 10 Jul. 2020, over 12 million cases of COVID-19 (in accordance with the applied case definitions and testing strategies in the affected countries) have been reported, including over 500,000 deaths.

COVID-19 is caused by a strain of coronavirus called severe acute respiratory syndrome coronavirus 2 (SARS-CoV-2). SARS-CoV-2 was previously referred to by its provisional name, 2019 novel coronavirus (2019-nCoV), and has also been called human coronavirus 2019 (HCoV-19 or hCoV-19).

SARS-CoV-2 is a positive-sense, single-stranded RNA virus that is contagious in humans. Taxonomically, SARS-CoV-2 is a strain of severe acute respiratory syndrome-related coronavirus (SARSr-CoV). It is believed to have zoonotic origins and has close genetic similarity to bat coronaviruses, suggesting it emerged from a bat-borne virus. The virus shows little genetic diversity, indicating that the spillover event introducing SARS-CoV-2 to humans is likely to have occurred in late 2019.

Epidemiological studies estimate that each COVID-19 infection results in 1.4 to 3.9 new ones when no members of the community are immune and no preventive measures taken. The virus is thought to primarily spread between people through close contact and via respiratory droplets produced from coughs or sneezes. It mainly enters human cells by binding to the receptor angiotensin converting enzyme 2 (ACE2).

Given the high rate of infectivity and mortality associated with SARS-CoV-2, there is clearly a need for sensitive, and reliable tests to determine whether a subject has been infected with SARS-CoV-2 and presently has anti-SARS-CoV-2 antibodies in their bloodstream. The present disclosure provides a biological sample assay for SARS-CoV-2 with enhanced accuracy in determining whether or not a subject is seropositive or seronegative for SARS-CoV-2, as well as related kits. In particular embodiments, the biological sample comprises serum, and in particular embodiments, the biological sample comprises saliva.

### Biological Sample Assay

The biological sample assays disclosed herein may be used to determine if a subject has antibodies specific for SARS-CoV-2.

In one aspect, the disclosure provides a method of determining whether a subject is seropositive or seronegative for SARS-CoV-2. The method may be used for the purposes of determining whether the subject had been infected with SARS-CoV-2. In particular embodiments, the assay may be a flow cytometry-based biological sample assay that identifies the presence in the subject's biological sample (e.g., serum, blood, or saliva) antibodies that bind SARS-CoV-2 antigens.

The methods comprise the use of an antibody-containing sample from a subject whose serostatus for SARS-CoV-2 is to be determined. Serostatus refers to the status (e.g., presence or absence) of a serological marker (e.g., SARS-CoV-2-specific antibodies) in a subject's blood, wherein a seropositive serostatus is the presence of detectable levels of a specific marker and a seronegative serostatus is the absence of detectable levels of a specific marker. For convenience and because of their similarity, “serum” and “plasma” are collectively referred to herein as serum. The presence and abundance of serum antibodies may correlate with the presence and abundance of antibodies present within biological samples other than serum and may act as a surrogate measurement to determine the serostatus of a subject. The terms “antibody-containing sample” and “biological sample” are used interchangeably herein to refer to an antibody-containing biological sample, which includes, but is not limited to, solutions within blood (e.g., serum and plasma), salivary secretions (e.g., saliva), lacrimal secretions (e.g., tears), respiratory secretions (e.g., mucus), and intestinal secretions (e.g., mucus). In one embodiment, the subject may be a mammal. In another embodiment, the subject may be a human. In some embodiments, the biological sample may be serum or plasma obtained from a blood draw from the subject. In some embodiments, the biological sample may be salivary secretions obtained from the subject. In some embodiments, the biological sample may be saliva obtained from the subject. In some embodiments, the biological sample may be lacrimal secretions obtained from the subject. In some embodiments, the biological sample may be tears obtained from the subject. In some embodiments, the biological sample may be respiratory secretions obtained from the subject. In some embodiments, the biological sample may be respiratory mucous obtained from the subject. In some embodiments, the biological sample may be intestinal secretions obtained from the subject. In some embodiments, the biological sample may be intestinal mucus obtained from the subject. The sample may also be an antibody-enriched sample such as an ammonium sulfate precipitate from a blood or other sample, or from a dried, e.g., lyophilized, biological sample.

In one aspect, the assay comprises: (a) incubating a biological sample obtained from a subject (e.g., serum, blood or saliva) with fixed and permeabilized test cells comprising: (i) cells infected with SARS-CoV-2 (positive test cells); and (ii) cells not infected with SARS-CoV-2 (negative test cells), wherein the positive test cells and the negative test cells are distinguishable from each other; (b) incubating the biological samples with a detection antibody that binds to antibodies in the biological sample; and (c) analyzing the test cell-biological sample admixture with an appropriate instrument capable of determining an amount of antibodies bound to the positive test cells and an amount of antibodies bound to the negative test cells, thereby determining whether the subject is seropositive for SARS-CoV-2. Optionally, the assay may comprise pre-adsorbing the biological sample to one or more populations of fixed and permeabilized control cells, e.g., cells infected with a pathogen other than SARS-CoV-2, to remove background and/or cross-reactive antibodies. In certain embodiments, the biological sample comprises serum, blood, or saliva.

Contact is maintained under conditions and for a time period sufficient for antibodies (e.g., SARS-CoV-2-specific antibodies) within the biological sample that immunoreact with antigens present in the test cells (e.g., SARS-CoV-2-specific antigens) and/or control cells to bind said antigens. This contact and maintenance is also referred to herein as incubation. In one embodiment, the time period may range from a few minutes to about 96 hours. In another embodiment, the time period may be about 1 to about 8 hours. In another embodiment, the time period may be about 2 to about 6 hours. In certain embodiments, the biological sample comprises serum, blood, or saliva.

In certain embodiments, the biological sample is incubated with a mixture of the positive test cells and the negative test cells. For example, in one embodiment, a biological sample obtained from a subject is admixed with a mixture of test cells comprising (a) cells uninfected with SARS-CoV-2 virus (negative test cells) and (b) cells infected with SARS-CoV-2 virus (positive test cells), thus allowing antibodies present in the biological sample to contact. (a) antigens present in negative test cells, and (b) antigens present in the positive test cells.

In certain embodiments, biological subsamples are independently incubated with different test cells, wherein one biological subsample is incubated with the positive test cells, and another biological subsample is incubated with the negative test cells, and wherein cells infected with SARS-CoV-2 and cells not infected with SARS-CoV-2 are not mixed together during this incubation step. For example, in one embodiment, a biological sample obtained from a subject is divided into at least two biological subsamples, wherein one biological subsample is admixed with a cell population comprising cells uninfected with SARS-CoV-2, and the other biological subsample is admixed with a cell population comprising cells infected with SARS-CoV-2 virus, thus allowing antibodies present in the biological subsamples to contact: (a) antigens present in the uninfected cells, or (b) antigens present in the SARS-CoV-2-infected cells. In certain embodiments, the biological sample comprises serum, blood, or saliva.

Following incubation with the test cells, the bound antibodies (e.g., physical matrix-bound (immunoreacted) antibodies) are separated from any unbound antibodies (e.g., non-bound (unreacted) antibodies) present in the reacted biological sample. In certain embodiments, this separation may be carried out by centrifugation and decantation or pipetting off the supernatant liquid, pipette removal as where the antigen-bound antibody is on the walls of a culture plate, elution, and the like. In certain embodiments, the biological sample or biological subsamples are removed from the bound antibodies and stored for potential re-testing. In certain embodiments, the biological sample comprises serum, blood, or saliva.

In certain embodiments, the biological sample is incubated with test cells (as discussed above), the biological sample is then removed from bound antibodies (e.g., matrix-bound (immunoreacted) antibodies), and then the bound antibodies are washed to remove residual biological sample, wherein the biological sample comprises remaining antibodies from the subject that are unbound to the test cells. In one embodiment, the wash solution may comprise Phosphate Buffered Saline (PBS). In another embodiment, the wash solution may comprise PBS-Tween20. In another embodiment, the wash solution may comprise Tris Buffered Saline (TBS). In another embodiment, the wash solution may comprise TBS-Tween20. In another embodiment, the wash solution may comprise Tris-HCl. In another embodiment, the wash solution may comprise Tris-HCl-Tween20. In another embodiment, the wash solution may comprise phosphate buffer (PB). In another embodiment, the wash solution may comprise alkaline phosphate buffer (AP). In another embodiment, the wash solution may comprise distilled water.

Washing is performed under conditions and for a time period sufficient to remove biological samples and any remaining antibodies of the biological sample that did not immunoreact, or bind, with the antigens present in either the positive or negative test cells. In one embodiment, the time period of washing may range from about 1 to about 15 minutes, or about 5 to about 15 minutes or more, and in certain embodiments, washing may be repeated about 1 to about 3 times or more. In another embodiment, the time period may be about 1 minute, about 2 minutes, about 3 minutes, or about 5 minutes, and the washing may be repeated about 3 times.

In certain embodiments, the washed test cells (as described above) are then contacted with a detection antibody, wherein the detection antibody comprises a detectable label, e.g., a labeled anti-human antibody or anti-human IgG antibody. Each of the test cells, having been exposed to the biological sample, are admixed and contacted with the detection antibody, e.g., labeled anti-human antibodies, to form labeled antigen-antibody complexes. Contact with the detection antibody is maintained for a time period sufficient for the detection antibody to bind antibodies within the biological sample that are bound to antigens within the test cells. In one embodiment, the time period may range from a few minutes to about 96 hours. In another embodiment, the time period may be about 1 to about 8 hours. In another embodiment, the time period may be about 2 to about 6 hours. In certain embodiments, the biological sample comprises serum, blood, or saliva.

The amount of immunoreaction between antibodies present within the biological sample and the test cells is determined for the biological sample following secondary labeling, e.g., with the anti-human antibodies that are admixed with the test cells. In certain embodiments, the biological sample comprises serum, blood, or saliva.

Detection antibodies are secondary antibodies deployed as tools for enabling qualitative and quantitative measurements respective to a target of interest by proxy of the label's ability to be measured in some capacity (also referred to as a reporter). In one embodiment, the detection antibody may be a labeled anti-human antibody. In another embodiment, the detection antibody may be a labeled anti-human IgG. In another embodiment, the detection antibody may be a labeled anti-human IgG, Fcy fragment specific. In another embodiment, the detection antibody may be a labeled anti-human IgA. In another embodiment, the detection antibody may be a labeled anti-human IgD. In another embodiment, the detection antibody may be a labeled anti-human IgM. In another embodiment, the detection antibody may be a labeled anti-human antibody fragment, such as an antigen-binding fragment (Fab), single chain variable fragment (scFV), or other antibody fragment or “third generation” (3G) antibody.

In certain embodiments, the detection antibody may be an anti-IgG antibody, and anti-human IgG where the subject whose biological sample is assayed is human, but many other antibodies may be used. The detection antibody is also labeled with a detectable marker. The detectable marker(s) labeling the test cells and the detectable marker labeling the detection antibody should be distinguishable from each other. In certain embodiments, the detectable marker is a fluorescent molecule. In certain embodiments, the fluorescent molecule labeling the detection antibody should have a fluorescence emission spectrum that does not overlap with the fluorescence emission spectrum of the fluorescent molecule used to label specific test cells (e.g., negative test cells uninfected with SARS-CoV-2 and positive test cells infected with SARS-CoV-2). In certain embodiments, the fluorescent molecule labeling the detection antibody may be allophycocyanin (APC), but many other fluorophores are suitable as well.

The label of the labeled anti-human antibody can be one of fluorescent tags, or colorimetric tags, wherein the tag is conjugated to the anti-human antibody and detectable upon irradiation, typically with defined wavelengths of light in the ultraviolet, visible, or infrared range (200-800 nm), and can be detected by appropriate instrumentation (e.g., flow cytometer, plate reader, or microfluidics chip). The label for the anti-human antibodies may be a covalently-linked fluorescent or chromogenic compound whose fluorescence emission spectrum or color does not overlap with the fluorescence emission spectrum or color of the colorant used to differentially label (i) uninfected and (ii) SARS-CoV-2-infected test cells. In one embodiment, the label of the labeled anti-human antibody may be a fluorescent tag. In another embodiment, the label of the labeled anti-human antibody may be allophycocyanin (APC). In another embodiment, the label of the labeled anti-human antibody may be phycoerythrin (PE). In another embodiment, the label of the labeled anti-human antibody may be tetramethylrhodamine isothiocyanate (TRITC). In another embodiment, the label of the labeled anti-human antibody may be peridinin chlorophyll protein (PerCP). In another embodiment, the label of the labeled anti-human antibody may be a colorimetric tag. In addition to using a fluorescent label for the anti-human (secondary) antibodies, enzyme labels such as horseradish peroxidase (HRP), alkaline phosphatase, and glucose oxidase may be covalently conjugated to the secondary antibodies as are often utilized in ELISA assays with an appropriate chromogenic substrate as are well known.

The anti-human (secondary) detection antibodies may themselves raised in an animal other than a human. Illustrative secondary antibodies can include those raised in goats, donkeys, horses, rabbits, mice and rats. In one embodiment, anti-human antibodies may react with human Fc antibody portions.

Any unreacted antibodies from the application of the detection antibodies may be separated from the immunoreaction products as previously discussed.

Cells

In certain embodiments, the test cells and/or control cells are Vero cells, but the disclosure contemplates the use of other types of cells, including but not limited to any of the cell types described herein. In one embodiment, the test cells, e.g., the negative test cells uninfected with SARS-CoV-2 virus and the positive test cells infected with SARS-CoV-2 virus, may be the same cell type to minimize possible differing cross-reactivities. In another embodiment, the control cells may be the same cell type as the test cells to minimize possible differing cross-reactivities. Illustrative cell types that may be used include but are not limited to: 1) monkey CV-1 kidney cells (Vero cells); 2) human SK-N-SH neuroblastoma cells; 3) human U2OS osteosarcoma cells: 4) human 293 embryonic kidney cells; 5) monkey COS cells; 6) mouse 3T3 cells; 7) hamster BHK-21 cells; 8) bovine BIEC cells; 9) bovine BUVEC cells; 10) human Caco-2 cells; 11) human HeLa cells, 12) monkey MA 104 cells; 13) canine MDCK cells, 14) pig PK-15 cells; and 15) human WiDr cells.

Furthermore, the positive test cells infected with SARS-CoV-2 virus may be generated by contacting desired cells with a sufficient amount of SARS-CoV-2 virions to generate infection. Similarly, control cells infected with a virus other than SARS-CoV-2 virus may be generated by contacting desired cells with a sufficient amount of the other virions to generate infection. Likewise, control cells infected with pathogens other than SARS-CoV-2 virus, or other viruses, may be generated by contacting desired cells with a sufficient amount of the other pathogens, e.g, bacteria, or parasites.

The test cells and/or control cells may themselves be attached to a solid, physical matrix (e.g., plastic dish, magnetic beads, agarose, etc.) or may be suspended in a liquid solution such as an aqueous medium like a buffer solution such as PBS.

In certain embodiments, the test cells and/or control cell with which the biological sample is contacted may be fixed and permeabilized cells, which allows antibodies present in the biological sample access to intracellular proteins, epitopes, including those present on SARS-CoV-2 virus present in the test cells or other pathogen present in the control cells. In one embodiment, slurries of fixed and permeabilized cells uninfected with SARS-CoV-2 and fixed and permeabilized cells infected with SARS-CoV-2 may be used. In certain embodiments, the biological sample comprises serum, blood, or saliva.

As is well known in the biological arts, cell fixation and permeabilization can be achieved by a wide variety of chemicals including, but not limited to treatment with one or more of formaldehyde, paraformaldehyde, methanol, ethanol, and acetone. Preparing test cells for evaluation using the assay and methods described herein may further include manually fixing, permeabilizing, and adding exogenously-introduced fluorescent colorant to cells.

Fixation may be used to preserve the biological samples from decay and may increase the mechanical strength or stability for further biochemical assessment Various fixation mechanisms are well known in the art and may be deployed to fix test cells used in the disclosed assay and methods. In one embodiment, formaldehyde may be used to fix test cells and/or control cells.

Permeabilization modifies the cell membrane to allow access of molecules, such as antibodies, across the cell membrane to better evaluate and detect intracellular antigens Various permeabilization mechanisms may be deployed to permeabilize test cells used in the disclosed assay and methods and are well known in the art. In one embodiment, methanol may be used to permeabilize test cells.

In certain embodiments, the test cells and/or antigens within the test cells and/or control cells are present within or attached to a physical matrix, so that the antibodies that bind to antigens within the test cells and/or control cells form physical matrix-bound antibodies (also referred to as matrix-bound antibodies). Illustrative physical matrices include, for example, 1) a protein-coated solid matrix (e.g., ELISA plate); 2) a cell-coated solid matrix (e.g., culture plate coated with fixed cells); 3) free-floating particles (e.g., live or fixed cells in liquid suspension); 4) a column of particles (e.g., live or fixed cells in a capillary tube); 5) protein-coated magnetic beads: 6) a slurry of protein-coated matrix (e.g., antigen-reacted CNBr-activated Sepharose® 4B) suspended in liquid, or packed into a flow-through column.

In certain embodiments, one or both of the positive test cells infected with SARS-CoV-2 and the negative test cells not infected with SARS-CoV-2 are labeled, e.g., with a detectable label, such as a fluorescent molecule. In certain embodiments, the negative test cells not infected with SARS-CoV-2 are labeled. In certain embodiments, the positive test cells infected with SARS-CoV-2 are labeled. In certain embodiments, both the positive test cells infected with SARS-CoV-2 and the negative test cells not infected with SARS-CoV-2 are labeled, e.g, with different labels, or by different degree of labeling using the same label, so the two cell populations can be distinguished from each other. In particular embodiments, the label is a fluorescent molecule. In another embodiment, the fluorescent molecule may be carboxyfluorescein diacetate succinimidyl ester (CFSE), but the disclosure contemplates the use of other detectable labels, including but not limited to other fluorophores described herein.

To distinguish between the test cells (e.g, between uninfected cells and cells infected with SARS-CoV-2 virus), an exogeneous (not normally present as part of the cells) detectable marker, such as a cellular colorant, may be used. In one embodiment, one of the two test cell populations may comprise a cellular colorant that is fluorescent upon irradiation, typically with defined wavelengths of light in the ultraviolet, visible, or infrared range (200-800 nm), and its fluorescence can be detected by a flow cytometer. In another embodiment, each of the two types of test cells may comprise a different cellular colorant that is fluorescent upon irradiation, such that the two types of test cells can be distinguished from each other. Useful exogenously-provided chemically reactive (covalently-linkable) fluorescent colorants include, but are not limited to, colorants that couple to amino groups such as epsilon-amino groups of lysine residues via N-hydroxysuccinimide ester exchange, and colorants that are chloromethyl reactive. In one embodiment, the colorant may be 5- (and 6)-carboxyfluorescein diacetate succinimidyl ester (CFSE). In another embodiment, the fluorescent colorant may be (CellTracer™ Violet). In another embodiment, the fluorescent colorant may be 2,5-dioxopyrrolidin-1-yl-7-(2-(((1E,3E,4E)-1,5-dichloro-6-oxohexa-1,4-dien-3-ylidene)amino)-5-hydroxyphenyl)octanoate (CellTrace™ Far Red DDAO-SE). In another embodiment, the fluorescent colorant may be (2,3,6,7-tetrahydro-9-bromomethyl-IH,5H-quinolizino(9,1-gh)-coumarin (CellTracker™ Violet BMQC). In another embodiment, the fluorescent colorant may be 7-amino-4-chloromethyl-coumarin (CellTracker™ Blue CMAC). In another embodiment, the fluorescent colorant may be 5-chloromethyl-fluorescein diacetate (CellTracker™ Green). In another embodiment, the fluorescent colorant may be 5-chloromethylrhodamine (CellTracker™ Red).

Test cells can also be differentially labeled with one or more intracellularly-expressed fluorescent proteins. In one embodiment, the fluorescent protein may be green fluorescent protein (GFP). In another embodiment, the fluorescent protein may be mCherry. In another embodiment, the fluorescent protein may be tdTomato. In another embodiment, the fluorescent protein may be KeimaRed. In another embodiment, the fluorescent protein may be yellow fluorescent protein (YFP). In another embodiment, the fluorescent protein may be cyan fluorescent protein (CFP). Labeling can include, but is not limited to, the above intracellularly-expressed fluorescent proteins. Such proteins examples are discussed for GFP in Chalfie et al, (1994) Science 263:802-805.

The exogenously-introduced fluorescent colorant provides a means by which the two types of test cells are distinguishable from each other by fluorescence. Fluorescence emission from the exogenously-provided cellular colorant of the two types of test cells are distinguishable from the fluorescence emission of any secondary antibodies discussed herein, and fluorescence of any other material present in the assay. In one embodiment, the SARS-CoV-2-infected cell populations may be pre-treated with the fluorescent colorant and the uninfected cell populations are not. In another embodiment, the uninfected cell populations may be pre-treated with the fluorescent colorant and the SARS-CoV-2-infected cell populations are not. In another embodiment, the SARS-CoV-2-infected cell populations and the uninfected cell populations may both be pre-treated with two different and distinguishable fluorescent colorants.

Of the test cells, the purpose of the uninfected negative test cells is to serve as a negative control, as these cells do not present with SARS-CoV-2 antigens. If the antibodies from a patient's biological sample contain SARS-CoV-2-specific antibodies, the SARS-CoV-2 antibodies will not bind with the uninfected cells, since they do not comprise SARS-CoV-2 antigens. In certain embodiments, the biological sample comprises serum, blood, or saliva.

In addition, the purpose of the SARS-CoV-2-infected positive test cells is to serve as an indicator of the presence of SARS-CoV-2-specific antibodies, as these cells present with SARS-CoV-2 antigens. If the antibodies from a patient's biological sample contain SARS-CoV-2-specific antibodies, they will bind with SARS-CoV-2 antigens in the SARS-CoV-2-infected cells. Conversely, if the antibodies from a patient's biological sample do not contain SARS-CoV-2-specific antibodies, there will be little to no antibody binding of SARS-CoV-2-specific antigens. In certain embodiments, the biological sample comprises serum, blood, or saliva.

Measuring Antibody Binding to Cells

Any method of appropriate detection for immunofluorescence or color may be used to determine which, if any, of the biological sample bound to the test cells including but not limited to fluorescent microscopy, a fluorescent plate reader, a flow cytometer, or a fluorescence-activated cell sorter. In some embodiments, a flow cytometer or FACS may be utilized as such machines can measure both (1) the differential fluorescent color that indicated whether the test cell antigen-containing matrix were uninfected cells, or cells infected with SARS-CoV-2 virus, and the instrument simultaneously measures (2) a second fluorescent color that is indicative of the primary variable under study; namely, the amount of human antibody bound to negative test cells uninfected with SARS-CoV-2 virus versus positive test cells infected with SARS-CoV-2. In certain embodiments, the biological sample comprises serum, blood, or saliva.

An instrument capable of gauging signal or report of detection antibodies can be any device capable of quantitatively measuring the fluorescence associated with individual test cells. In certain embodiments, the instrument capable of gauging signal t of detection antibodies may be a flow cytometer capable of evaluating cells of an appropriate diameter, wherein the appropriate diameter is about 1 to about 20 microns. Examples of such appropriately sized cells from test cells include, but are not limited to, (1) live uninfected Vero cells, (2) fixed and permeabilized uninfected Vero cells, (3) live SARS-CoV-2-infected Vero cells, and (4) fixed and permeabilized SARS-CoV-2-infected Vero cells. In another embodiment, the cell sorting device may be a flow cytometer, but a fluorescence-activated cell sorter (FACS) can be used for the same purpose.

Samples from the subject exhibiting greater immunoreaction of biological sample antibodies to cells infected with SARS-CoV-2 virus compared to the immunoreaction of biological sample antibodies to cells uninfected with SARS-CoV-2 virus indicates a seropositive result for SARS-CoV-2.

Assay results indicating a low-positive result may be false-positive. Low-positive results may be the result of cross-reactive antibodies for SARS-CoV-2 present within the subject's biological sample. A biological sample believed to potentially contain cross-reactive antibodies can be pre-adsorbed against cells infected with pathogens other than SARS-CoV-2, wherein any potentially cross-reactive antibodies present in the biological sample may react with antigens in cells other than SARS-CoV-2-infected cells. A biological sample that has undergone pre-adsorption may have a depleted level of potentially cross-reactive antibodies, allowing for increased sensitivity of the assay. In certain embodiments, a biological sample may undergo pre-adsorption prior to making contact with test cells.

In certain embodiments, analysis of the immunofluorescence of the test cells can reveal whether or not the subject has been infected by SARS-CoV-2 based on the presence of SARS-CoV-2 antibodies bound to SARS-CoV-2-infected positive test cells. By determining the level to which test cells, uninfected or infected with SARS-CoV-2, the antibodies present in the biological sample bound, one can thereby ascertain whether the patient has been infected with SARS-CoV-2 or not. For example, results showing a greater amount of the biological sample antibodies bound the SARS-CoV-2-infected positive test cells compared to the uninfected negative test cells, indicates that the subject is seropositive for SARS-CoV-2 However, if there is no significant difference between the amount of biological sample antibodies that bound the SARS-CoV-2-infected positive test cells compared to uninfected negative test cells, indicates that the subject is seronegative for SARS-CoV-2.

The interpretation of the SARS-CoV-2 antibody assay comprises evaluating the amount of biological sample antibodies that bound the cells infected with SARS-CoV-2 virus compared to the cells uninfected with SARS-CoV-2 virus. When the test cell types (uninfected and infected with SARS-CoV-2) are analyzed in a mixture where both cells are present, they must be distinguished. Further, the amount of detection antibody must be measured on both test cell types and comparatively analyzed. Any method of appropriate detection for immunofluorescence or color may be used to determine which, if any, of the antibodies from the biological sample bound to the test cells including but not limited to fluorescent microscopy, a fluorescent plate reader, an absorbance plate reader, a flow cytometer, a microfluidics chip, or a fluorescence-activated cell sorter. In certain embodiments, e.g., when the assay is performed using a plate reader, the positive and negative test cells are assayed in different wells.

In certain embodiments, a flow cytometer or FACS may be utilized, as such machines can simultaneously measure both: (1) the differential fluorescent color that indicates whether the test cells are uninfected cells or cells infected with SARS-CoV-2 virus; and (2) a second fluorescent color that indicates the amount of human antibody bound to test cells uninfected with SARS-CoV-2 virus versus test cells infected with SARS-CoV-2 virus. Appropriate excitation is applied to the test cells wherein the test cells are categorically analyzed based on the appropriate label used to differentiate the cells uninfected with SARS-CoV-2 virus from the cells infected with SARS-CoV-2 virus. Additional excitation is applied to the test cells wherein the secondary antibody is detected. Mean fluorescence intensity is then calculated per event basis for both the cells uninfected with SARS-CoV-2 virus and the cells infected with SARS-CoV-2 virus.

In one embodiment, designating between a seropositive test result, a seronegative test result, and a low-positive test result may be determined by statistically defined means, wherein a population of SARS-CoV-2 seropositive subjects and a population of SARS-CoV-2 seronegative subjects are initially evaluated using the SARS-CoV-2 antibody assay as disclosed herein, and the test results of the populations are used to determine positive and negative test result ranges and parameters, thereby establishing statistical distributions and ranges that may be used to interpret test results and determine the serostatus of patients. In certain embodiments, the test result for each subject is defined as the detection antibody signal or report measured on positive test cells normalized to the detection antibody signal or report measured on negative test cells. For example, it may be defined as positive test cell signal divided by negative test cell signal (i.e., normalized test result). In some embodiments, the results may be used to define ranges for clear positive status and clear negative status, and/or to identify overlapping ranges that may be considered “low positive” and warrant additional testing. In certain embodiments, a subject is determined to be seronegative if the subject's normalized test result is less than or within 2 standard deviations of the mean normalized test result determined for a population of SARS-CoV-2 seronegative subjects. In certain embodiments, a subject is determined to be seropositive if the subject's normalized test result is greater than or within 2 standard deviations of the mean normalized test result determined for a population of SARS-CoV-2 seropositive subjects. In certain embodiments, a subject is determined to be a “low positive” if the normalized test result falls within an overlapping range that is within 2 standard deviations of the mean normalized test result determined for a population of SARS-CoV-2 seronegative subjects and also within 2 standard deviations of the mean normalized test result determined for a population of SARS-CoV-2 seropositive subjects.

In certain embodiments, determining SARS-CoV-2 serostatus using the SARS-CoV-2 antibody assay as disclosed herein may be achieved by comparing the detection antibody signal or report determined for a patient (e.g., the patient's normalized test result) to a defined value, e.g., a defined positive value and/or a defined negative value (e.g., normalized positive and/or negative values). In particular embodiments, the defined value may be a predetermined value, such as a value previously determined based on prior testing of a plurality of seropositive and/or seronegative subjects. In other embodiments, the defined value may be a calculated value, e.g., a value determined at the same time as the patient's value is determined, based on values generated using a plurality of positive and/or negative control biological samples. In certain embodiments, the biological sample comprises serum, blood, or saliva.

Thus, in certain embodiments, a subject is determined to be seropositive if the subject's detection antibody signal or report is above or within two or three standard deviations of a defined positive value. In certain embodiments, the signals or report and the defined positive value are normalized based on the ratio of the result on positive test cells to the result on negative test cells.

In certain embodiments, a patient's value is determined as the ratio of the subject's detection antibody signal or report measured on positive test cells to the subject's detection antibody signal or report measured on negative test cells. For example, it may be defined as positive test cell signal divided by negative test cell signal (i.e., normalized test result).

In certain embodiments, a subject is determined to be seropositive if the subject's detection antibody signal or report is greater than a defined negative value in a statistically significant manner. In certain embodiments, a subject is determined to be seropositive if the subject's detection antibody signal or report is greater than a defined negative value plus two or three standard deviations, i.e., greater than two or three standard deviations above a defined negative value. In certain embodiments, a subject is determined to be seropositive if the subject's detection antibody signal or report is at least 2-fold, at least 3-fold, or at least 5-fold greater than a defined negative value. In certain embodiments, a subject is determined to be seropositive if the subject's detection antibody signal or report is at least 2-fold, at least 3-fold, or at least 5-fold greater than two or at least three standard deviations of a defined negative value. In each case, the subject's detection antibody signal or report and the defined negative value may be normalized values.

In certain embodiments, a predetermined positive value is defined as an average normalized level of detection antibody signal or report and standard deviation determined for a plurality of subjects infected with SARS-CoV-2 (e.g., as determined using both positive test cells and negative test cells). In particular embodiments, a pre-determined positive value is the minimum value for a seropositive test result from positive test cells. In certain embodiments, the pre-determined positive value may be a value that is statistically (e.g., significantly) greater than the average detection antibody signal or report of the assay negative control standards. In other embodiments, the pre-determined positive value may be a value measuring at least two standard deviations greater than the average normalized detection antibody signal or report of the assay negative control standards. In another embodiment, the pre-determined positive value may be a value measuring at least three standard deviations greater than the average normalized detection antibody signal or report of the assay negative control standards. In another embodiment, the subject is determined seropositive for SARS-CoV-2, if the normalized detection antibody signal or report is at least 3-fold, at least 4-fold, or at least 5-fold greater in positive test cells infected with SARS-CoV-2 virus than in negative test cells uninfected with SARS-CoV-2 virus.

Concentrations of antibodies in a biological sample may be variable. In cases where antibody concentrations are low, wherein detection antibody signal or report from test cells are lower than the assay control standards or the detection antibody signal or report from positive test cells is present yet less than a pre-determined positive value, evaluating serostatus using a pre-determined positive value may be inappropriate. In such instances, a calculated positive value may be used to aid in determining serostatus. In certain embodiments, establishing defined values for SARS-CoV-2 serostatus, using the SARS-CoV-2 antibody assay as disclosed herein, may be achieved by determining the level of detection antibody signal or report (e.g., experimental value) from a biological sample from a subject (e.g., using both positive test cells and negative test cells), wherein a calculated positive value (e.g., the minimum value for a seropositive test result) can be generated. In certain embodiments, the calculated positive value may be a value that is determined to be statistically (e.g., significantly) greater from positive test cells compared to negative test cells (e.g., level of detection antibody signal or report). In general, if the analysis produces results indicating that the detection antibody signal or report is greater, e.g., significantly greater, in the positive test cells infected with SARS-CoV-2 virus as compared to the negative test cells uninfected with SARS-CoV-2 virus, then the biological sample is identified as seropositive for SARS-CoV-2. Alternatively, if the analysis produces results indicating that the detection antibody signal or report is not greater in the positive test cells infected with SARS-CoV-2 virus compared to the negative test cells uninfected with SARS-CoV-2 virus, the biological sample is identified as seronegative for SARS-CoV-2. In certain embodiments, the biological sample comprises serum, blood, or saliva.

In certain embodiments, if the analysis produces results indicating that the mean fluorescence intensity is greater (e.g., significantly greater) in the positive test cells infected with SARS-CoV-2 virus compared to the negative test cells uninfected with SARS-CoV-2 virus, the biological sample is identified as seropositive for SARS-CoV-2. In another embodiment, if the analysis produces results indicating that the mean fluorescence intensity is not greater in the positive test cells infected with SARS-CoV-2 virus compared to the negative test cells uninfected with SARS-CoV-2 virus, the biological sample is identified as seronegative for SARS-CoV-2.

In certain embodiments, the subject is determined seropositive for SARS-CoV-2, if the detection antibody signal or report from positive test cells infected with SARS-CoV-2 virus is greater than or equal to a pre-determined positive value. In another embodiment, the subject is determined seropositive for SARS-CoV-2, if the detection antibody signal or report from positive test cells infected with SARS-CoV-2 virus is greater than or equal to a calculated positive value. In another embodiment, the subject is determined seropositive for SARS-CoV-2, if the detection antibody signal or report is at least 3-fold, at least 4-fold, or at least 5-fold greater in positive test cells infected with SARS-CoV-2 virus than in negative test cells uninfected with SARS-CoV-2 virus.

In certain embodiments, the subject is determined seropositive for SARS-CoV-2, if the mean fluorescence intensity from positive test cells infected with SARS-CoV-2 virus is greater than or equal to a pre-determined positive value, or within two or three standard deviations of the pre-determined positive value. In another embodiment, the subject is determined seropositive for SARS-CoV-2 if the mean fluorescence intensity from positive test cells infected with SARS-CoV-2 virus is greater than or equal to a calculated positive value compared to negative test cells uninfected with SARS-CoV-2 virus, or greater than two or three standard deviations above a pre-determined negative value. In certain embodiments, the subject is determined seropositive for SARS-CoV-2, if the mean fluorescence intensity is at least 3-fold greater in the positive test cells infected with SARS-CoV-2 virus than in the negative cells uninfected with SARS-CoV-2 virus. In another embodiment, the subject is determined seropositive for SARS-CoV-2 if the mean fluorescence intensity is at least 3-fold, at least 4-fold, or at least 5-fold greater in the positive test cells infected with SARS-CoV-2 virus than in the negative test cells uninfected with SARS-CoV-2 virus. In particular embodiments of any of the calculations, the mean fluorescence intensity is normalized, and the calculated positive value and/or calculated negative value is also normalized.

In certain embodiments, the subject is determined seronegative for SARS-CoV-2, if the detection antibody signal or report is not statistically greater in the positive test cells infected with SARS-CoV-2 virus than in the negative test cells uninfected with SARS-CoV-2 virus. In certain embodiments, the subject is determined seronegative for SARS-CoV-2, if the detection antibody signal or report is less than 2%, less than 5%, less than 10%, or less than 15% greater in the positive test cells infected with SARS-CoV-2 virus than in the negative test cells uninfected with SARS-CoV-2 virus. In certain embodiments, the subject is determined seronegative for SARS-CoV-2, if the detection antibody signal or report is not statistically greater in the positive test cells infected with SARS-CoV-2 virus than in the negative test cells uninfected with SARS-CoV-2 virus. In another embodiment, the subject is determined seronegative for SARS-CoV-2, if the detection antibody signal or report is less than a pre-determined positive value in positive test cells infected with SARS-CoV-2 virus. In another embodiment, the subject is determined seronegative for SARS-CoV-2, if the detection antibody signal or report is less than a calculated positive value in positive test cells infected with SARS-CoV-2 virus compared to negative test cells uninfected with SARS-CoV-2 virus. In particular embodiments, the mean fluorescence intensity is normalized, and the calculated negative value is also normalized.

In certain embodiments, the subject is determined seronegative for SARS-CoV-2, if the mean fluorescence intensity is not statistically greater in the positive test cells infected with SARS-CoV-2 virus than in the negative test cells uninfected with SARS-CoV-2 virus. In another embodiment, the subject is determined seronegative for SARS-CoV-2, if the mean fluorescence intensity is less than a pre-determined positive value in positive test cells infected with SARS-CoV-2 virus. In another embodiment, the subject is determined seronegative for SARS-CoV-2, if the mean fluorescence intensity is less than a calculated positive value in positive test cells infected with SARS-CoV-2 virus compared to negative test cells uninfected with SARS-CoV-2 virus. In certain embodiments of all cases, the values compared are normalized.

Low-positive results may be the result of SARS-CoV-2 cross-reactive antibodies present in the subject's biological sample. In certain embodiments, a biological sample is determined as possibly containing SARS-CoV-2 cross-reactive antibodies, if the detection antibody signal or report of positive test cells infected with SARS-CoV-2 is at least or about 1%, 2%, 3%, 4%, 5%, 6%, 7%, 8%, 9%, 10%, 20%, 30%, 40%, 50%, 75%, 100%, 150%, 200% or more, but not more than either a pre-determined positive value or a calculated positive value compared to negative test cells uninfected with SARS-CoV-2. In certain embodiments, where a biological sample is determined as possibly containing SARS-CoV-2 cross-reactive antibodies, a biological sample from the subject is reevaluated, e.g., using methods incorporating a pre-adsorption step as disclosed herein. The values may be normalized.

In certain embodiments, a biological sample is determined as possibly containing SARS-CoV-2 cross-reactive antibodies if the mean fluorescence intensity of positive test cells infected with SARS-CoV-2 is at least or about 1%, 2%, 3%, 4%, 5%, 6%, 7%, 8%, 9%, 10%, 20%, 30%, 40%, 50%, 75%, 100%, 150%, 200% or more, but not more than either a pre-determined positive value or a calculated positive value compared to negative test cells uninfected with SARS-CoV-2, wherein the subject should be reevaluated using methods incorporating a pre-adsorption step as mentioned herein.

Tests may be repeated to verify initial results. In certain embodiments, tests repeated to verify initial results may utilize methods incorporating a pre-adsorption step as mentioned herein. In certain embodiments, if the detection antibody signal or report of positive test cells infected with SARS-CoV-2 is greater than, but no greater than a pre-determined positive value or a calculated positive value compared to negative test cells uninfected with SARS-CoV-2, a biological sample may be reevaluated using methods incorporating a pre-adsorption step as mentioned herein. In another embodiment, if the mean fluorescence intensity of positive test cells infected with SARS-CoV-2 is greater than, but no greater than a pre-determined positive value or a calculated positive value compared to negative test cells uninfected with SARS-CoV-2, a biological sample may be reevaluated using methods incorporating a pre-adsorption step as mentioned herein. In certain embodiments, if the detection antibody signal or report of positive test cells infected with SARS-CoV-2 is more than a pre-determined positive value or a calculated positive value compared to negative test cells uninfected with SARS-CoV-2, a biological sample may be reevaluated using methods incorporating a pre-adsorption step as mentioned herein. In another embodiment, if the mean fluorescence intensity of positive test cells infected with SARS-CoV-2 is more than a pre-determined positive value or a calculated positive value compared to negative test cells uninfected with SARS-CoV-2, a biological sample may be reevaluated using methods incorporating a pre-adsorption step as mentioned herein.

Biological sample antibodies not specific to SARS-CoV-2 may potentially result in detection antibody signal or report artifact, wherein signal or report interference renders results that may be difficult to determine or interpret. In certain embodiments, subjects with auto-immune diseases may produce test results comprising detection antibody signal, or report, artifact due to biological sample antibodies binding to non-SARS-CoV-2 epitopes within both the SARS-CoV-2 uninfected negative test cells and the SARS-CoV-2-infected positive test cells (e.g., anti-nuclear antibodies). In another embodiment, a biological sample, from a subject with an auto-immune disease, with a seropositive test result may be reevaluated using methods incorporating a pre-adsorption step as mentioned herein.

The assay may further include a calibrating solution respective to the type of biological sample being analyzed. In some embodiments, the assay may further include a calibrating solution comprising a calibrating biological sample or calibrating sample-mimic. In some embodiments, the assay may further include a calibrating solution comprising a calibrating serum or serum-mimic. In some embodiments, the assay may further include a calibrating solution comprising a calibrating salivary secretion or salivary secretion-mimic. In some embodiments, the assay may further include a calibrating solution comprising a calibrating saliva or saliva-mimic. In some embodiments, the assay may further include a calibrating solution comprising a calibrating a lacrimal secretion or lacrimal secretion-mimic. In some embodiments, the assay may further include a calibrating solution comprising a calibrating tear or tear-mimic. In some embodiments, the assay may further include a calibrating solution comprising a calibrating respiratory secretion or respiratory secretion-mimic. In some embodiments, the assay may further include a calibrating solution comprising a calibrating intestinal secretion or intestinal secretion-mimic. The calibrating solution may be used to gauge test results (e.g., subject's biological sample antibodies binding test cell antigens), in which the test results are normalized against the calibrating solution. Use of a calibrator may aid in determining a seropositive or seronegative test result.

The assay may further include the use of biological sample controls. In certain embodiments, biological sample controls may individually comprise: i) antibodies specific to SARS-CoV-2 (positive biological sample control); or ii) biological sample free of SARS-CoV-2-specific antibodies (negative biological sample control). In certain embodiments, biological sample control incubated with test cells may be used to validate the efficacy of the assay. For example, positive biological sample control comprising SARS-CoV-2-specific antibodies should result in a positive test result, as binding of said antibodies should occur with antigens present in SARS-CoV-2-infected cells; reciprocally, negative biological sample control comprising antibody-free biological sample should result in a negative test result, as no binding should occur with antigens present in SARS-CoV-2-infected cells. In certain embodiments, the biological sample control comprises serum, blood, or saliva.

Results are for the detection of SARS-CoV-2 antibodies. IgG antibodies to SARS-CoV-2 are generally detectable in blood several days after initial infection, although the duration of time antibodies are present post-infection is not well characterized. Individuals may have detectable virus present for several weeks or longer following seroconversion.

The sensitivity of the SARS-CoV-2 antibody assay early after infection is unknown. Negative results early after infection do not preclude acute SARS-CoV-2 infection. If acute infection is suspected, direct testing for SARS-CoV-2 by reverse transcription-polymerase chain reaction (RT-PCR) may be warranted.

### Further Embodiments of Assay Methods

Antibodies other than SARS-CoV-2-specific antibodies may be present in the biological sample. Antibodies other than SARS-CoV-2-specific antibodies can manifest within a subject through infection by pathogens other than SARS-CoV-2. Antibodies other than SARS-CoV-2-specific antibodies may operate in a cross-reactive manner. Cross-reactivity is a phenomenon by which an antibody raised against a specific antigen has a high affinity for a different antigen, commonly by result of similar structural epitopes between antigens. Consequently, antibodies not specific to SARS-CoV-2 might interfere with the evaluation of the presence of SARS-CoV-2-specific antibodies via cross-reactivity, wherein antibodies not specific to SARS-CoV-2 bind with antigens present in SARS-CoV-2-infected positive test cells. Subjects that have been infected by pathogens other than SARS-CoV-2, such as various viruses, bacteria, and parasites may have cross-reactive antibodies within their biological sample. In one embodiment, the infection of one or more of pathogens similar to that of SARS-CoV-2, e.g., a different coronavirus, may result in a biological sample comprising cross-reactive antibodies. In another embodiment, the infection of one or more of pathogens dissimilar to that of SARS-CoV-2 may result in a biological sample comprising cross-reactive antibodies. In another embodiment, the infection of one or more of influenza A virus, influenza B virus, hepatitis C virus, hepatitis B virus, Haemophilus influenza virus, alpha coronavirus 229E, alpha coronavirus NL63, beta coronavirus OC43, beta coronavirus HKU1, or respiratory syncytial virus may result in a biological sample comprising cross-reactive antibodies. In certain embodiments, the biological sample comprises serum, blood, or saliva.

Biological samples comprising antibodies that are SARS-CoV-2 cross-reactive may yield low-positive results utilizing the assays and methods disclosed. In cases of low-positive results, where cross-reactivity is suspected, a subject's biological sample may be tested again. In one embodiment, a freshly derived biological sample or a stored aliquot of the original biological sample may be used. Similarly, even without receiving low-positive results utilizing the assays and methods disclosed, the biological sample may undergo a pre-adsorption step prior to contacting the biological sample with the test cells. In one embodiment, a biological sample that has not yet been evaluated using the assays and methods disclosed may undergo a pre-adsorption step. In another embodiment, a biological sample that has been evaluated using the assays and methods disclosed may be reevaluated using a freshly derived biological sample, or a stored aliquot of the original biological sample may be evaluated by the methods disclosed herein that include a pre-adsorption step. In certain embodiments, the biological sample comprises serum, blood, or saliva.

Biological samples comprising, or believed to comprise, SARS-CoV-2 cross-reactive antibodies, as well as biological samples not believed to comprise SARS-CoV-2 cross-reactive antibodies may undergo pre-adsorption prior to contacting the biological sample with test cells comprising cells uninfected with SARS-CoV-2 virus and cells infected with SARS-CoV-2 virus. Pre-adsorption comprises contacting the biological sample with fixed and permeabilized cells exhibiting antigens that may bind SARS-CoV-2 cross-reactive antibodies to deplete any SARS-CoV-2 cross-reactive antibodies from the biological sample and recollecting the pre-adsorbed biological sample to evaluate using test cells comprising negative test cells uninfected with SARS-CoV-2 virus and positive test cells infected with SARS-CoV-2 virus. In the pre-adsorption process, the biological sample is contacted with one or more cells (i.e., control cells) infected with a pathogen or pathogens that potentially exhibit SARS-CoV-2 cross-reactive antigens. In certain embodiments, a panel of cells, each infected with one or more different pathogens that potentially exhibit SARS-CoV-2 cross-reactive antigens may be used. In another embodiment, the pre-adsorption control cells are infected with one or more pathogens other than SARS-CoV-2, wherein non-SA RS-Cov-2 pathogens include, but are not limited to, various viruses, bacteria, and parasites. In another embodiment, the pre-adsorption control cells may be infected with one or more pathogens similar to SARS-CoV-2, such as other coronaviruses. In another embodiment, the pre-adsorption control cells may be infected with SARS-CoV-2. In another embodiment, the pre-adsorption control cells may be infected with one or more pathogens dissimilar to SARS-CoV-2. In another embodiment, the pre-adsorption control cells may be infected with one or more pathogens both similar and dissimilar to SARS-CoV-2. In another embodiment, the pre-adsorption control cells may be infected with one or more of influenza A virus, influenza B virus, hepatitis C virus, hepatitis B virus, Haemophilus influenza virus, alpha coronavirus 229E, alpha coronavirus NL63, beta coronavirus OC43, beta coronavirus HKU1, respiratory syncytial virus, or any combination thereof.

In certain embodiments, the biological sample that undergoes pre-adsorption prior to immunoreaction with test cells, e.g., a freshly derived biological sample or aliquot from the original biological sample, can be individually exposed to one or more control cells, e.g., (i) uninfected cells, or (ii) non-SARS-CoV-2 pathogen-infected cells. Biological sample pre-adsorbed with uninfected cells may help to remove antibodies other than SARS-CoV-2-specific antibodies. Biological sample pre-adsorbed against non-SARS-CoV-2 pathogen-infected cells may help to remove antibodies other than SARS-CoV-2-specific antibodies and potentially SARS-CoV-2 cross-reactive antibodies. If the subject's biological sample has SARS-CoV-2 cross-reactive antibodies, those antibodies present in the subject's biological sample will bind to the pre-adsorption control cells as applicable. The pre-adsorbed biological sample, comprising depleted non-SARS-CoV-2-specific antibodies, and/or depleted SARS-CoV-2 cross-reactive antibodies, may then be collected for evaluation.

As a result of the pre-adsorptions, a pre-adsorbed biological sample is formed. Pre-adsorbed biological samples incubated with pre-adsorption control cells infected with pathogens other than SARS-CoV-2 may contain a reduced amount of antibodies that potentially immunoreact with SARS-CoV-2 antigens. Biological samples that undergo pre-adsorption thereby contain a relatively enhanced amount of antibodies that immunoreact with SARS-CoV-2 antigens specifically, when those antibodies are present in the biological sample. Therefore, pre-adsorption may be used to increase the efficacy of test results and/or validate initial test results.

Biological samples that undergo pre-adsorption may also be used to potentially determine previous infection with other pathogens. For example, low-positive primary test results (for SARS-CoV-2) that later produce negative secondary test results (for SARS-CoV-2) after using a pre-adsorption step where the control cell population was infected with a singular specific pathogen (other than SARS-CoV-2), may imply that the subject is seropositive for said specific pathogen used to infect the control cell population.

In certain embodiments, a biological sample assay method for determining whether a subject is seropositive for SARS-CoV-2 or not, comprises the following steps in order: 1) admixing a biological sample obtained from the subject with test cells, wherein the biological sample comprises antibodies from the subject, and wherein the test cells comprise: (i) fixed and permeabilized test cells uninfected with SARS-CoV-2 virus; and (ii) fixed and permeabilized test cells uninfected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a first admixture comprising the biological sample and the fixed and permeabilized test cells, and maintaining said first admixture under conditions and for a time period sufficient to permit the antibodies to bind to antigens present in the fixed and permeabilized test cells; 2) removing the biological sample from said first admixture, wherein the biological sample comprises remaining antibodies from the subject unbound to the fixed and permeabilized test cells; 3) admixing a detection antibody with the fixed and permeabilized test cells, wherein the detection antibody comprises a detectable label, thereby generating a second admixture comprising the detection antibody and the fixed and permeabilized test cells, and maintaining said second admixture for a time period sufficient to permit the detection antibody to bind to the antibodies bound to the fixed and permeabilized test cells; 4) washing said second admixture to remove unbound detection antibody from the fixed and permeabilized test cells; 5) measuring: (i) an amount of detection antibody bound to the fixed and permeabilized cells uninfected with SARS-CoV-2 virus; and (ii) an amount of detection antibody bound to the fixed and permeabilized cells infected with SARS-CoV-2 virus; and 6) conclusively determining whether the subject is seropositive for SARS-CoV-2 or not, wherein if the amount of the detection antibody measured in SARS-CoV-2-infected test cells is equivalent to or greater than a pre-determined positive value or a calculated positive value compared to the amount of detection antibody measured in cells uninfected with SARS-CoV-2, the subject is determined seropositive for SARS-CoV-2. In certain embodiments, the biological sample comprises serum, blood, or saliva.

In certain embodiments, a biological sample assay method for determining whether a subject is seropositive for SARS-CoV-2 or not, comprises the following steps in order: 1) admixing a biological sample obtained from the subject with test cells, wherein the biological sample comprises antibodies from the subject, and wherein the test cells comprise: (i) fixed and permeabilized test cells uninfected with SARS-CoV-2 virus; and (ii) fixed and permeabilized test cells uninfected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a first admixture comprising the biological sample and the fixed and permeabilized test cells, and maintaining said first admixture under conditions and for a time period sufficient to permit the antibodies to bind to antigens present in the fixed and permeabilized test cells; 2) removing the biological sample from said first admixture, wherein the biological sample comprises remaining antibodies from the subject unbound to the fixed and permeabilized test cells; 3) admixing a detection antibody with the fixed and permeabilized test cells, wherein the detection antibody comprises a detectable label, thereby generating a second admixture comprising the detection antibody and the fixed and permeabilized test cells, and maintaining said second admixture for a time period sufficient to permit the detection antibody to bind to the antibodies bound to the fixed and permeabilized test cells; 4) washing said second admixture to remove unbound detection antibody from the fixed and permeabilized test cells; 5) measuring: (i) an amount of detection antibody bound to the fixed and permeabilized cells uninfected with SARS-CoV-2 virus; (ii) an amount of detection antibody bound to the fixed and permeabilized cells infected with SARS-CoV-2 virus; 6) inconclusively determining whether the subject is seropositive for SARS-CoV-2 or not, due to low-positive results; 7) pre-adsorbing a biological sample by admixing a new freshly derived biological sample, or aliquot from the original biological sample, obtained from the same patient with fixed and permeabilized control cells comprising pathogen antigens other than SARS-CoV-2-specific antigens, thereby generating a third admixture, and maintaining said third admixture for a time period sufficient to permit the cross-reactive antibodies in the biological sample to bind to the pathogen antigens present in the fixed and permeabilized control cells; 8) collecting the pre-adsorbed biological sample from the third admixture, wherein the pre-adsorbed biological sample has been depleted of SARS-CoV-2 cross-reactive antibodies; 9) admixing the pre-adsorbed biological sample with test cells, wherein the pre-adsorbed biological sample comprises antibodies from the subject depleted of SARS-CoV-2 cross reactive antibodies, and wherein the test cells comprise: (i) fixed and permeabilized test cells uninfected with SARS-CoV-2 virus; and (ii) fixed and permeabilized test cells uninfected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a fourth admixture comprising the pre-adsorbed biological sample and the fixed and permeabilized test cells, and maintaining said forth admixture under conditions and for a time period sufficient to permit the antibodies to bind to antigens present in the fixed and permeabilized test cells; 10) removing the pre-adsorbed biological sample from said fourth admixture, wherein the pre-adsorbed biological sample comprises remaining antibodies from the subject unbound to the fixed and permeabilized test cells; 11) admixing a detection antibody with the fixed and permeabilized test cells, wherein the detection antibody comprises a detectable label, thereby generating a fifth admixture comprising the detection antibody and the fixed and permeabilized test cells, and maintaining said fifth admixture for a time period sufficient to permit the detection antibody to bind to the antibodies bound to the fixed and permeabilized test cells; 12) washing said fifth admixture to remove unbound detection antibody from the fixed and permeabilized test cells; 13) measuring: (i) an amount of detection antibody bound to the fixed and permeabilized cells uninfected with SARS-CoV-2 virus; (ii) an amount of detection antibody bound to the fixed and permeabilized cells infected with SARS-CoV-2 virus; and 14) conclusively determining whether the subject is seropositive for SARS-CoV-2 or not, wherein if the amount of the detection antibody measured in SARS-CoV-2-infected test cells is equivalent to or greater than a pre-determined positive value or a calculated positive value compared to the amount of detection antibody measured in cells uninfected with SARS-CoV-2, the subject is determined seropositive for SARS-CoV-2. In certain embodiments, the biological sample comprises serum, blood, or saliva.

In certain embodiments, a biological sample assay method for determining whether a subject is seropositive for SARS-CoV-2 or not, comprises the following steps in order: 1) pre-adsorbing a biological sample by admixing a biological sample obtained from a subject with fixed and permeabilized control cells comprising pathogen antigens other than SARS-CoV-2-specific antigens, thereby generating a first admixture, and maintaining said first admixture for a time period sufficient to permit the cross-reactive antibodies in the biological sample to bind to the pathogen antigens present in the fixed and permeabilized control cells; 2) collecting the pre-adsorbed biological sample from the first admixture, wherein the pre-adsorbed biological sample has been depleted of SARS-CoV-2 cross-reactive antibodies; 3) admixing the pre-adsorbed biological sample with test cells, wherein the pre-adsorbed biological sample comprises antibodies from the subject depleted of SARS-CoV-2 cross reactive antibodies, and wherein the test cells comprise: (i) fixed and permeabilized test cells uninfected with SARS-CoV-2 virus; and (ii) fixed and permeabilized test cells uninfected with SARS-CoV-2 virus, wherein the fixed and permeabilized test cells uninfected with SARS-CoV-2 virus and the fixed and permeabilized test cells infected with SARS-CoV-2 virus are distinguishable from each other, thereby generating a second admixture comprising the pre-adsorbed biological sample and the fixed and permeabilized test cells, and maintaining said second admixture under conditions and for a time period sufficient to permit the antibodies to bind to antigens present in the fixed and permeabilized test cells; 4) removing the pre-adsorbed biological sample from said fourth admixture, wherein the pre-adsorbed biological sample comprises remaining antibodies from the subject unbound to the fixed and permeabilized test cells; 5) admixing a detection antibody with the fixed and permeabilized test cells, wherein the detection antibody comprises a detectable label, thereby generating a third admixture comprising the detection antibody and the fixed and permeabilized test cells, and maintaining said third admixture for a time period sufficient to permit the detection antibody to bind to the antibodies bound to the fixed and permeabilized test cells; 6) washing said third admixture to remove unbound detection antibody from the fixed and permeabilized test cells; 7) measuring: (i) an amount of detection antibody bound to the fixed and permeabilized cells uninfected with SARS-CoV-2 virus; (ii) an amount of detection antibody bound to the fixed and permeabilized cells infected with SARS-CoV-2 virus; and 8) conclusively determining whether the subject is seropositive for SARS-CoV-2 or not, wherein if the amount of the detection antibody measured in SARS-CoV-2-infected test cells is equivalent to or greater than a pre-determined positive value or a calculated positive value compared to the amount of detection antibody measured in cells uninfected with SARS-CoV-2, the subject is determined seropositive for SARS-CoV-2. In certain embodiments, the biological sample comprises serum, blood, or saliva.

In certain embodiments, pre-adsorption steps may be repeated multiple times with multiple control cells infected with various non-SARS-CoV-2 pathogens, including other members of the Coronaviridae family, which may share a high degree of structural similarity with SARS-CoV-2 epitopes, to insure removal of potential SARS-CoV-2 cross-reactive antibodies.

Methods disclosed herein describe the antibody binding to virus-infected cells assay using a mixture of test cells comprising cells uninfected with SARS-CoV-2 virus and cells infected with SARS-CoV-2 virus. Alternatively, test cells comprising cells uninfected with SARS-CoV-2 virus and cells infected with SARS-CoV-2 virus may be analyzed separately from each other, wherein the test cells are not mixed together.

The patient (subject) sample (i.e., biological sample) is divided into at least two portions or subsamples (i.e., biological subsamples). Each portion (subsample) is separately admixed and contacted with populations of fixed and permeabilized cells uninfected with SARS-CoV-2 virus, and populations of fixed and permeabilized cells infected with SARS-CoV-2 virus, wherein the biological sample comprising antibodies of the subject will contact (a) antigens from uninfected cells, and (b) antigens from SARS-CoV-2-infected cells. Remaining steps mimic that of the methods previously described utilizing a single biological sample and mixed test cells comprising cells uninfected with SARS-CoV-2 virus and cells infected with SARS-CoV-2 virus, wherein the biological subsamples are removed from the test cell populations after incubation and optionally retained, followed by washing of the test cell populations, incubation with a detection antibody, additional washing of the test cell populations, and subsequent evaluation of biological sample antibodies bound to test cell populations by measurement of detection antibody.

In certain embodiments, when the test cell populations (uninfected cells and cells infected with SARS-CoV-2 virus), are kept separate from each other, they remain distinguishable without the need to be labeled in any fashion, although labeling can still be used to further aid in distinguishing the test cell populations.

Similar to the methods previously described utilizing a single biological sample and mixed test cells comprising cells uninfected with SARS-CoV-2 virus and cells infected with SARS-CoV-2 virus, test results yielding low-positive results, where cross-reactivity is suspected, biological subsamples may need to undergo further evaluation utilizing pre-adsorption.

The previously described methods utilize fixed and permeabilized cells for the SARS-CoV-2 antibody assay, however the same principles can be applied to cell lysates to evaluate the SARS-CoV-2 serostatus of a patient.

### Biological Sample Assay Kit

The present invention also discloses a biological sample assay kit for carrying out an assay disclosed herein. In particular embodiments of any of the kits disclosed herein, the assay is a serological assay. The disclosure further includes a biological sample assay kit for determining whether a subject is seropositive for SARS-CoV-2, or not. In certain embodiments, the kit comprises vessels separately containing i) test cells uninfected with SARS-CoV-2 virus, and (ii) test cells infected with SARS-CoV-2 virus. The test cells are, or are to be, differentially labeled with a fluorophore, or fluorophores, such that a cell sorting device or flow cytometer can differentiate each of the two populations. Additionally, test cells are, or are to be, fixed and permeabilized.

In certain embodiments, the kit comprises one or more vessels containing pre-adsorption (i.e., control) cells infected with one or more of pathogens other than SARS-CoV-2. The pre-adsorption control cells may be provided in a variety of forms.

In certain embodiments, the kit comprises a vessel containing secondary detection antibodies. In certain embodiments, secondary detection antibodies comprise labeled anti-human antibodies. In another embodiment, the label of the anti-human antibodies may be a fluorescent material whose fluorescence is distinguishable from the fluorescence of any other material present. In another embodiment, subject's biological sample may be combined with the test cells provided to determine the relative abundance of SARS-CoV-2 specific antibody in a cell sorting device or flow cytometer. In certain embodiments, each of vessels may contain a sufficient amount of the recited ingredient to carry out at least one assay. In another embodiment, the instructions for carrying out an assay may also be present in the kit.

One illustrative kit includes: a) a vessel that contains i) uninfected cells, and (ii) SARS-CoV-2-infected cells, wherein these test cells are distinguishable from each other by fluorescence emission when irradiated with wavelengths of light that excite an exogenously-introduced fluorescent colorant. The exogenously-introduced fluorescent colorant by which each type of test cells (e.g. cells uninfected with SARS-CoV-2 virus, and cells infected with SARS-CoV-2 virus) is distinguishable from each other by the intensity of fluorescent emissions in a defined wavelength, and is also distinguishable from any other fluorescent species utilized in the assay. In one embodiment, the SARS-CoV-2-infected cells may be labeled by exogenously-introduced fluorescent colorant and the uninfected cells are not labeled. In another embodiment, the uninfected cells may be labeled by the exogenously-introduced fluorescent colorant and the SARS-CoV-2-infected cells are not labeled. In another embodiment, the SARS-CoV-2-infected cells and the uninfected cells may both be labeled by different and distinguishable exogenously-introduced fluorescent colorants.

A second vessel may also be included in the kit. The second vessel contains control cells infected with a pathogen, or pathogens, other than SARS-CoV-2.

The kit may further include a third vessel that contains labeled anti-human antibodies. The label of the anti-human antibodies is a fluorescent material whose fluorescence is distinguishable from the fluorescence of any other material present. In one embodiment, the label of the labeled anti-human antibodies may comprise an antibody-linked fluorescent molecule or fluorophore. In another embodiment, the label and antibody of the labeled anti-human antibodies may comprise allophycocyanin (APC) conjugated anti-human IgG.

The kit may further include a fourth vessel that contains a biological sample calibrating solution respective to the type of biological sample being analyzed. The calibrating solution may be used to gauge test results (e.g. subject's biological sample antibodies binding test cell antigens), in which the test results are normalized against the calibrating solution. In some embodiments, the calibrating solution may comprise a biological sample or biological sample-mimic. In some embodiments, the calibrating solution may comprise a serum or serum-mimic. In some embodiments, the calibrating solution may comprise a salivary secretion or salivary secretion-mimic. In some embodiments, the calibrating solution may comprise a saliva or saliva-mimic. In some embodiments, the calibrating solution may comprise a lacrimal secretion or lacrimal secretion-mimic. In some embodiments, the calibrating solution may comprise a respiratory secretion or respiratory secretion-mimic. In some embodiments, the calibrating solution may comprise a intestinal secretion or intestinal secretion-mimic. Use of a calibrator may aid in determining a seropositive or seronegative test result. In certain embodiments, the biological sample comprises serum, blood, or saliva.

The kit may further include a fifth and a sixth vessel individually containing controls, e.g., biological sample controls. Biological sample controls may individually comprise i) biological sample comprising antibodies specific to SARS-CoV-2; and ii) antibody-free biological sample. In certain embodiments, biological sample control incubated with test cells may be used to validate the efficacy of the assay. For example, biological sample control comprising SARS-CoV-2-specific antibodies should result in a positive test result as binding of said antibodies should occur with antigens present in SARS-CoV-2-infected cells; reciprocally biological sample control comprising antibody-free biological sample, should result in a negative test result as no binding should occur with antigens present in SARS-CoV-2-infected cells. In certain embodiments, the biological sample control comprises serum, blood, or saliva.

Each of the vessels may contain a sufficient amount of the recited ingredient to carry out at least one assay.

Another illustrative kit includes: a) two separate vessels that separately contain one of i) uninfected cells, and (ii) SARS-CoV-2-infected cells, wherein these test cells are distinguishable from each other by fluorescence emission when irradiated with wavelengths of light that excite an exogenously-introduced fluorescent colorant. The exogenously-introduced fluorescent colorant by which each type of test cells (e.g cells uninfected with SARS-CoV-2 virus, and cells infected with SARS-CoV-2 virus) is distinguishable from each other by the intensity of fluorescent emissions in a defined wavelength, and is also distinguishable from any other fluorescent species utilized in the assay. In one embodiment, the SARS-CoV-2-infected cells may be labeled by exogenously-introduced fluorescent colorant and the uninfected cells are not labeled. In another embodiment, the uninfected cells may be labeled by the exogenously-introduced fluorescent colorant and the SARS-CoV-2 cells are not labeled. In another embodiment, the SARS-CoV-2-infected cells and the uninfected cells may both be labeled by different and distinguishable exogenously-introduced fluorescent colorants.

A third vessel may also be included in the kit. The third vessel contains test cells infected with a pathogen, or pathogens, other than SARS-CoV-2.

The kit may further include a fourth vessel that contains labeled anti-human antibodies. The label of the anti-human antibodies is a fluorescent material whose fluorescence is distinguishable from the fluorescence of any other material present. In one embodiment, the label of the labeled anti-human antibodies may comprise an antibody-linked fluorescent molecule or fluorophore. In another embodiment, the label and antibody of the labeled anti-human antibodies may comprise allophycocyanin (APC) conjugated anti-human IgG.

The kit may further include a fifth vessel that contains a calibrating solution respective to the type of biological sample being analyzed. In certain embodiments, the fifth vessel comprises calibrating serum or serum mimic.

The kit may further include a sixth and a seventh vessel individually containing controls, e.g., Biological sample controls. Biological sample controls may individually comprise: i) a biological sample comprising antibodies specific to SARS-CoV-2; and ii) an antibody-free biological sample. In certain embodiments, the control is a serum control.

Each of the vessels may contain a sufficient amount of the recited ingredient to carry out at least one assay.

The vessels of a contemplated assay kit may be made of glass or a plastic to which the recited reagents adhere poorly, such as to polyethylene glycol (PEG) coatings and coatings of polytetrafluoroethylene (PTFE).

Instructions for carrying out an assay may also present in the kit. A contemplated kit may be provided as a container that holds the recited components.

Additionally, the above-described biological sample assay kit may further include other components used in preparing and performing the assays and methods described in the disclosure Other contents of the biological sample assay kit may comprise:
Packing materials
Phosphate buffered saline (PBS)
37% formaldehyde
90% methanol
25 mg carboxyfluorescein diacetate succinimidyl ester (CFSE)
Dimethyl sulfoxide (DMSO)
2 mL serological pipette
2×5 mL serological pipette
2×10 mL serological pipette
1.7 mL centrifuge tubes
Donor Equine Serum (DES)
500 mL PBS
10% NaAzide
PBS-S
Donkey Gamma Globulin
Goat Gamma Globulin
25 mL serological pipette
50 mL conical tubes

Treating desired cells with an exogenously-induced colorant allows for appropriate distinction between test cell comprising cells uninfected with SARS-CoV-2 virus and cells infected with SARS-CoV-2 virus. Numerous cell colorants and intracellularly expressed proteins are provided above. In one embodiment, the exogenously-induced fluorescent colorant may comprise carboxyfluorescein succinimidyl ester (CFSE). Protocols for CFSE staining are well known in the art and should be applied to the assay and methods described herein to develop test cells exhibiting the characteristics produced by an exogenously-induced colorant.

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

The materials and methods employed in these experiments are now described.

### Example 1

### SARS-Cov-2 Antibody Assay

This example describes an illustrative embodiment of the methods disclosed herein for determining whether a subject has antibodies against SARS-CoV-2.

Sample Preparation and Assay: Whole blood is collected from the patient by venous blood draw using a red-top tube (or any appropriate blood collection tube that does not contain an anti-coagulant). After red blood cells have clotted they are removed from the sample via centrifugation, and 1 mL of remaining serum is used for evaluation.

A serum sample from a single patient is diluted and added to the well of a 96-well plate containing a mixture of fixed and permeabilized uninfected Vero test cells and (carboxyfluorescein succinimidyl ester) CFSE-labelled SARS-CoV-2-infected Vero test cells and incubated.

Uninfected test cells (which are not labelled with CFSE) are negative for the presence of SARS-CoV-2. Thus, SARS-CoV-2 specific antibodies, if present, from the patient's serum will not bind to the uninfected test cells.

SARS-CoV-2-infected test cells (which are labelled with CFSE) are positive for presence of SARS-CoV-2. Thus, SARS-CoV-2-specific antibodies, if present, from the patient's serum bind to SARS-CoV-2-specific antigens exhibited by SARS-CoV-2 test cells.

After test cells are incubated with serum samples, the serum samples are removed and the test cells are washed to remove any remaining serum comprising antibodies that did not immunoreact with test cells.

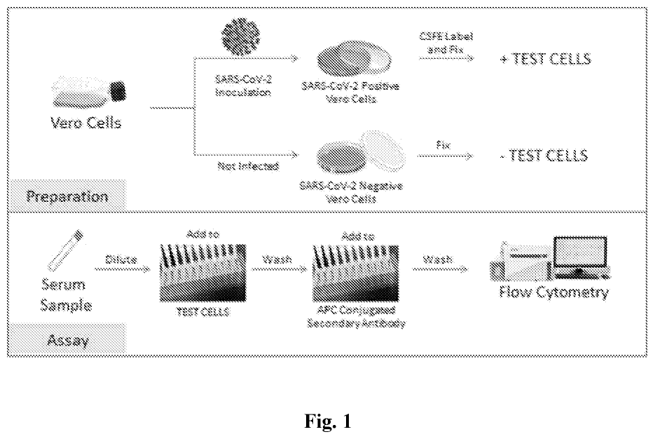

After washing, test cells are then incubated with allophycocyanin (APC) conjugated anti-human IgG (detection antibody). The APC labelled anti-human IgG binds to anti-SARS-CoV-2 (and any other) antibodies bound to the test cells comprising a mixed population of uninfected and SARS-CoV-2-infected cells, which can be distinguished by flow-cytometry (FIG. 1).

If SARS-CoV-2 specific antibodies are present within the patient's serum, they will bind with antigens exhibited specifically by SARS-CoV-2-infected test cells and not with uninfected test cells, which do not exhibit SARS-CoV-2-specific antigens. Thus, in situations where a patient's serum possesses SARS-CoV-2 antibodies, less APC anti-human IgG binds to antibodies that immunoreacted with uninfected test cells than that of SARS-CoV-2-infected test cells.

Reciprocally, if SARS-CoV-2 specific antibodies are absent from the patient's serum, no antibody binding will occur with SARS-CoV-2-specific antigens exhibited by SARS-CoV-2-infected cells. Because uninfected cells do not exhibit SARS-CoV-2-specific antigens, patient's serum comprising antibodies will not be capable of forming an immunoreaction between antibodies and SARS-CoV-2-specific antigens. Thus, in situations where a patient's serum lacks SARS-CoV-2 specific antibodies, less APC anti-human IgG binds to antibodies that immunoreacted with test cells. The test cell population is read by a flow-cytometer to distinguish sera from patients who do not have antibodies to SARS-CoV-2 from sera which is positive for the presence of SARS-CoV-2 antibodies. Flow cytometry is performed on a CytoFLEX (Beckman Coulter) flow cytometer running CytoFLEX CytExpert Software in accordance with the CytoFLEX instruction manual (IFU, version 2.3).

### Performing Flow Cytometry and Interpretation of Results:

Flow cytometry is executed on a benchtop. Surfaces are decontaminated by wiping with 70% ethanol solution. Personal protective equipment is worn in accordance with requirements of Good Laboratory Practices (GLP) and the Clinical Laboratory Improvement Amendment (CLIA) or its equivalent (e.g., COLA, CAP).

The SARS-CoV-2 IgG Test is conducted using flow cytometry performed on a CytoFLEX (Beckman Coulter) flow cytometer running CytoFLEX CytExpert Software in accordance with the CytoFLEX instruction manual (IFU, version 2.3). The flow cytometer is turned on and the system start-up is performed as described in the CytoFLEX instruction manual.

After system startup, CytExpert software is used and the plate layout is entered by selecting “Add Plate” from the dropdown menu. The plate type is “96 well U-bottom” and the wells that contain the samples are identified.

The SARS-CoV-2 IgG Test cells that have been incubated with serum and detection antibodies (the 96-well plates) are loaded into the flow cytometer.

The software is set to run samples using the following parameters:
Events to display: 10,000 events
Events to record: 10,000 events—in P1
Time to record: 600 seconds

A forward scatter vs. side scatter (FSC/SSC) dot plot is created and then the first sample well is run. A polygonal gate is then drawn around the cell population or “cloud” and labeled P1. A histogram plot is created that reads “FITC vs count” gated on P1. A dot plot, FITC-A (x-axis) vs. APC-A (y-axis) gated on P1 is created. A rectangular gate is drawn around each distinct population or “cloud”. The first gate is labeled P2.

The remainder of the samples are then read. Data from the run(s) will be saved and exported to a CSV/Excel File.

A statistical table for “All Events” for P2 populations is created using the following parameters:
Events
% Total
Mean Fluorescence Intensity (MFI) APC-A

For each serum sample, the Mean Fluorescence Intensity (MFI) APC data is used to compile the following: SARS-CoV-2 uninfected cells (P2 Mean APC) and SARS-CoV-2-infected cells (A).

The data is used to determine the SARS-CoV-2 serostatus for each serum sample. Results of testing are provided in reports that are either written or conveyed electronically as appropriate. In general, a positive result for evaluating the presence of SARS-CoV-2 antibodies from a patient's serum could be interpreted from visualizing statistically greater MFI from SARS-CoV-2-infected test cells compared to uninfected test cells. Conversely, in general, a negative result for evaluating the presence of SARS-CoV-2 antibodies from a patient's serum could be interpreted from visualizing similar MFI in both infected and SARS-CoV-2-infected test cells.

### Alternative Methods for Instances of Cross-Reaction Interference

In rare cases, patient serum may include antibodies with the potential to cross-react with the SARS-CoV-2 antigens. In such instances, the SARS-CoV-2 IgG Test would render false-positive results.

Examples that could result in a false-positive include but are not limited to one or more of the following:
anti-influenza A (IgG and IgM)
anti-influenza B (IgG and IgM)
anti-HCV (IgG and IgM)
anti-HBV (IgG and IgM)
anti-Haemophilus influenzae (IgG and IgM)
anti-229E (alpha coronavirus)
anti-NL63 (alpha coronavirus)
anti-OC43 (beta coronavirus)
anti-HKU1 (beta coronavirus)
anti-HIV
anti-respiratory syncytial virus (IgG and IgM)
ANA (antinuclear antibodies)

In rare cases with low-positive results, where cross-reactivity is suspected, patient samples are tested again.

During this retesting process, the patient's serum is pre-adsorbed against one or more populations of infected Vero cells to deplete any antibodies causing cross-reactivity. In the pre-adsorption process, manufactured Vero cells infected with a panel of potentially cross-reactive viruses, bacteria, or parasites including but not limited to those listed above are used.

Serum from patients with low-positive or suspected false-positive results from the SARS-CoV-2 IgG Test are individually exposed to these pre-adsorption control cells, depleting common antibodies as well as type-specific antibodies (not specific to SARS-CoV-2) that bind to the pre-adsorption control cells as applicable.

The depletion of potentially cross-reactive antibodies from the patient samples allows for unhindered assessment of SARS-CoV-2-infected Vero test cells. These pre-adsorbed serum samples generate a definitive result on the SARS-CoV-2 IgG Test while also potentially identifying the potential prior exposure resulting in cross-reactivity.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

What is claimed is:

1. A method for determining whether a subject is seropositive for SARS-CoV-2 or not, comprising (optionally in the following order):
a) admixing a biological sample obtained from the subject with test cells, wherein the biological sample comprises antibodies from the subject, and wherein the test cells comprise:
(i) fixed and permeabilized test cells uninfected with SARS-CoV-2 virus; and
(ii) fixed and permeabilized test cells infected with SARS-CoV-2 virus,
wherein the fixed and permeabilized test cells uninfected with SARS-CoV-2 virus and the fixed and permeabilized test cells infected with SARS-CoV-2 virus are distinguishable from each other, thereby generating a first admixture comprising the biological sample and the fixed and permeabilized test cells, and
maintaining said first admixture under conditions and for a time period sufficient to permit the antibodies to bind to antigens present in the fixed and permeabilized test cells;
b) removing the biological sample from said first admixture, wherein the biological sample comprises remaining antibodies from the subject unbound to the fixed and permeabilized test cells;
c) admixing a detection antibody with the fixed and permeabilized test cells, wherein the detection antibody comprises a detectable label, thereby generating a second admixture comprising the detection antibody and the fixed and permeabilized test cells, and maintaining said second admixture for a time period sufficient to permit the detection antibody to bind to the antibodies bound to the fixed and permeabilized test cells;
d) washing said second admixture to remove unbound detection antibody from the fixed and permeabilized test cells; and
e) measuring:
(i) an amount of detection antibody bound to the fixed and permeabilized cells uninfected with SARS-CoV-2 virus; and
(ii) an amount of detection antibody bound to the fixed and permeabilized cells infected with SARS-CoV-2 virus,
thereby determining whether the subject is seropositive for SARS-CoV-2 or not, wherein if the amount of detection antibody measured in (e)(ii) is greater than the amount of detection antibody measured in (e)(i), and the amount of detection antibody measured in (e)(ii) meets or exceeds a pre-determined positive value or a calculated positive value compared to the amount of detection antibody measured in (e)(i) the subject is determined seropositive for SARS-CoV-2.

2. The method of claim 1, wherein a biological sample comprises an antibody-containing biological sample.

3. The method of claim 2, wherein an antibody-containing biological sample comprises serum, blood, salivary secretions, lacrimal secretions, respiratory secretions, or intestinal secretions, optionally serum, blood, or saliva.

4. A method for determining whether a subject is seropositive for SARS-CoV-2 or not, comprising (optionally in the following order):
a) admixing a biological sample obtained from the subject with fixed and permeabilized control cells comprising pathogen antigens other than SARS-CoV-2-specific antigens, thereby generating a first admixture; and
maintaining said first admixture for a time period sufficient to permit the cross-reactive antibodies in the biological sample to bind to the pathogen antigens present in the fixed and permeabilized control cells, thereby experiencing pre-adsorption;
b) collecting the biological sample from the first admixture, wherein the biological sample has been depleted of SARS-CoV-2 cross-reactive antibodies;
c) admixing the biological sample with test cells, wherein the biological sample comprises antibodies from the subject depleted of SARS-CoV-2 cross-reactive antibodies, and wherein the test cells comprise:
(i) fixed and permeabilized test cells uninfected with SARS-CoV-2 virus; and
(ii) fixed and permeabilized test cells infected with SARS-CoV-2 virus,
wherein the fixed and permeabilized test cells uninfected with SARS-CoV-2 virus and the fixed and permeabilized test cells infected with SARS-CoV-2 virus are distinguishable from each other, thereby generating a second admixture comprising the biological sample and the fixed and permeabilized test cells, and
maintaining said second admixture under conditions and for a time period sufficient to permit the antibodies to bind to antigens present in the fixed and permeabilized test cells;
d) removing the biological sample from said second admixture, wherein the biological sample comprises remaining antibodies from the subject unbound to the fixed and permeabilized test cells;
e) admixing a detection antibody with the fixed and permeabilized test cells, wherein the detection antibody comprises a detectable label, thereby generating a third admixture comprising the detection antibody and the fixed and permeabilized test cells, and maintaining said third admixture for a time period sufficient to permit the detection antibody to bind to the antibodies bound to the fixed and permeabilized test cells;
f) washing said third admixture to remove unbound detection antibody from the fixed and permeabilized test cells; and
g) measuring:
(i) an amount of detection antibody bound to the fixed and permeabilized cells uninfected with SARS-CoV-2 virus; and
(ii) an amount of detection antibody bound to the fixed and permeabilized cells infected with SARS-CoV-2 virus,
thereby determining whether the subject is seropositive for SARS-CoV-2 or not, wherein if the amount of detection antibody measured in (g)(ii) is greater than the amount of detection antibody measured in (g)(i), and the amount of detection antibody measured in (g)(ii) meets or exceeds a pre-determined positive value or a calculated positive value compared to the amount of detection antibody measured in (g)(i) the subject is determined seropositive for SARS-CoV-2.

5. The method of claim 4, wherein a biological sample comprises an antibody-containing biological sample.

6. The method of claim 5, wherein an antibody-containing biological sample comprises blood, salivary secretions, lacrimal secretions, respiratory secretions, and intestinal secretions.

7. The method of claim 4, wherein the fixed and permeabilized control cells comprise cells infected with influenza A virus, influenza B virus, hepatitis C virus, hepatitis B virus, Haemophilus influenza virus, alpha coronavirus 229E, alpha coronavirus NL63, beta coronavirus OC43, beta coronavirus HKU1, or respiratory syncytial virus, or any combination thereof.

8. The method of claim 4, wherein the fixed and permeabilized control cells comprise antigens cross-reactive with SARS-CoV-2.

9. The method of any one of claims 1-8, wherein the fixed and permeabilized test cells uninfected with SARS-CoV-2 and the fixed and permeabilized test cells infected with SARS-CoV-2 are distinguishable from each other by fluorescence emission when irradiated with wavelengths of light that excite an exogenously-introduced fluorescent colorant.

10. The method of claim 9, wherein the exogenously-introduced fluorescent colorant comprises a dye, optionally an intracellularly fluorescent molecule.

11. The method of claim 9, wherein the exogenously-introduced fluorescent colorant forms a covalent linkage with intracellular molecules in the fixed and permeabilized test cells.

12. The method of claim 9, wherein said exogenously-introduced fluorescent colorant comprises caroxyfluorescein succinimidyl ester (CFSE).

13. The method of claim 9, wherein either one, but not both, of:
(i) the fixed and permeabilized test cells uninfected with SARS-CoV-2; or
(ii) the fixed and permeabilized cells test cell infected with SARS-CoV-2 comprise the exogenously-introduced fluorescent colorant.

14. The method of claim 9, wherein:
(i) the fixed and permeabilized test cells uninfected with SARS-CoV-2; and
(ii) the fixed and permeabilized test cells infected with SARS-CoV-2 each comprise a different exogenously-introduced fluorescent colorant by which the two fixed and permeabilized test cells are distinguishable from each other according to the intensity of fluorescence in a defined emission spectrum, and which is distinguishable from the fluorescence emission spectrum of any other fluorophores used in the assay.

15. The method of any one of claims 1-14, wherein the antigens present in the fixed and permeabilized test cells that bind the antibodies present in the biological sample are measured by incubating said antigens bound to antibodies with labeled anti-human antibodies to form labeled antigen-antibody complexes.

16. The method of claim 15, wherein said labeled anti-human antibodies are labeled with a compound whose fluorescence emission spectrum is distinguishable from the fluorescence emission spectrum of any other fluorophores utilized in the assay.

17. The method of claim 15, wherein the label of said labeled anti-human antibodies comprises an antibody-linked fluorescent molecule or fluorophore.

18. The method of claim 15, wherein said labeled anti-human antibodies comprises allophycocyanin (APC) conjugated anti-human IgG.

19. A biological sample assay kit for determining whether a subject is seropositive for SARS-CoV-2 or not according to the method of any one of claims 1-18, comprising:
a) one or more vessels collectively comprising:
(i) fixed and permeabilized test cells uninfected with SARS-CoV-2 virus; and
(ii) fixed and permeabilized test cells infected with SARS-CoV-2 virus,
wherein the fixed and permeabilized test cells uninfected with SARS-CoV-2 and the fixed and permeabilized test cells infected with SARS-CoV-2 are distinguishable from each other; and
b) a second vessel comprising:
(iii) fixed and permeabilized control cells comprising pathogen antigens other than SARS-CoV-2-specific antigens,

20. The biological sample assay kit of claim 19, wherein the fixed and permeabilized control cells are infected with one or more of: influenza A virus, influenza B virus, hepatitis C virus, hepatitis B virus, Haemophilus influenza virus, alpha corona virus 229E, alpha coronavirus NL63, beta coronavirus OC43, beta coronavirus HKU1, or respiratory syncytial virus, or any combination thereof.

21. The biological sample assay kit of claim 19, wherein the fixed and permeabilized control cells comprise antigens cross-reactive with SARS-CoV-2.

22. The biological sample assay kit of claim 19, further including a third vessel comprising labeled anti-human antibodies.

23. The biological sample assay kit of claim 22, wherein the label of said labeled anti-human antibodies comprises a compound that when irradiated provides a fluorescence emission spectrum distinguishable from that of any other fluorophores utilized in the assay.

24. The biological sample assay kit of claim 22, wherein the labeled anti-human antibodies comprise allophycocyanin (APC) conjugated anti-human IgG.

25. The biological sample assay kit of claim 19, further including a fourth vessel comprising a calibrating solution respective to the type of biological sample being analyzed, optionally wherein the biological sample comprises serum, blood, or saliva.

26. The biological sample assay kit of claim 25, wherein the calibrating solution comprises a calibrating biological sample or biological sample-mimic.

27. The biological sample assay kit of claim 25, wherein the calibrating solution comprises a serum or serum-mimic.

28. The biological sample assay kit of claim 25, wherein the calibrating solution comprises a saliva or saliva-mimic.

29. The biological sample assay kit of claim 19, further including a fifth vessel comprising a positive biological sample control.

30. The biological sample assay kit of claim 29, wherein the positive biological control comprises SARS-CoV-2-specific antibodies.

31. The biological sample assay kit of claim 19, further including a sixth vessel comprising a negative biological sample control.

32. The biological sample assay kit of claim 31, wherein the negative biological sample control comprises antibody-free biological sample.

33. The biological sample assay kit of claim 19, wherein the fixed and permeabilized test cells uninfected with SARS-CoV-2 and the fixed and permeabilized test cells infected with SARS-CoV-2 are distinguishable from each other by fluorescence emission when irradiated with wavelengths of light that excite an exogenously-introduced fluorescent colorant.

34. The biological sample assay kit of claim 33, wherein the exogenously-introduced fluorescent colorant comprises a dye, optionally an intracellularly fluorescent molecule.

35. The biological sample assay kit of claim 33, wherein the exogenously-introduced fluorescent colorant forms a covalent linkage with intracellular molecules in the fixed and permeabilized test cells.

36. The biological sample assay kit of claim 33, wherein said exogenously-introduced fluorescent colorant comprises caroxyfluorescein succinimidyl ester (CFSE).

37. The biological sample assay kit of any one of claims 19-36, wherein either one, but not both, of:
(i) the fixed and permeabilized test cells uninfected with SARS-CoV-2; or
(ii) the fixed and permeabilized test cells infected with SARS-CoV-2 comprise an exogenously-introduced fluorescent colorant.

38. The biological sample assay kit of any one of claims 19-36, wherein:
(i) the fixed and permeabilized test cells uninfected with SARS-CoV-2; and
(ii) the fixed and permeabilized test cells infected with SARS-CoV-2 each comprise a different exogenously-introduced fluorescent colorant by which the two fixed and permeabilized test cells are distinguishable from each other according to the intensity of fluorescence in a defined emission spectrum, and which is distinguishable from the fluorescence emission spectrum of any other fluorophores used in the assay.
